(19) United States
(12) Patent Application Publication
(10) Pub. No.: US 2022/0025324 A1
(43) Pub. Date: Jan. 27, 2022

(54) METHODS OF PRODUCING VENOUS ANGIOBLASTS AND SINUSOIDAL ENDOTHELIAL CELL-LIKE CELLS AND COMPOSITIONS THEREOF

(71) Applicant: University Health Network, Toronto (CA)

(72) Inventors: Gordon Keller, Toronto (CA); Blair Kenneth Gage, Toronto (CA)

(21) Appl. No.: 17/277,637

(22) PCT Filed: Sep. 18, 2019
(86) PCT No.: PCT/IB2019/057882

§ 371 Date: Mar. 18, 2021

Related U.S. Application Data
(60) Provisional application No. 62/732,934, filed on Sep. 18, 2018.

(51) Int. Cl. C12N 5/071

(52) U.S. Cl. CPC C12N 5/067; C12N 2501/165

(57) ABSTRACT

Disclosed herein are methods of producing a population of venous angioblast cells from stem cells using a venous angioblast inducing media and optionally isolating a CD34+ population from the cell population comprising the venous angioblast cells, for example using a CD34 affinity reagent, CD31 affinity reagent and/or CD144 affinity reagent, optionally with or without a CD73 affinity reagent as well as methods of further differentiating the venous angioblasts in vitro to produce SEC-LCs and/or in vivo to produce SECs. Uses of the cells and compositions comprising the cells are also described.

### CROSS-REFERENCE TO RELATED APPLICATIONS

This application claims priority to U.S. application Ser. No. 62/732,934, filed on Sep. 18, 2018. The disclosure of the prior application is considered part of the disclosure of this application, and is incorporated in its entirety into this application.

### FIELD

The invention relates to methods for producing venous endothelial lineage cells and, in particular, to sinusoidal endothelial cell-like cells, compositions that include such cells and uses thereof.

### INTRODUCTION

Diseases requiring transplantation including liver disease effects many people and transplantation with cadaveric organs is hampered by donor availability. While tissue engineering provides hope of lab designed donor tissue, it requires functional cell types to be used in its construction or at least functional progenitors capable of co-maturation to a therapeutically relevant tissue. Within normal liver tissue for example, multiple types of endothelial cell types are present and represent the interaction surface between hepatocytes and the rest of the body. Primarily, this interaction occurs within the sinusoidal vascular network across the Liver Sinusoidal Endothelial Cells (LSECs) through their dynamically controlled transcellular fenestrations arranged in sieve plates (Wisse et al., 1985). Different Sinusoidal Endothelial Cells (SECs) exist in addition to LSECs including Bone Marrow Sinusoidal Endothelial Cells (BSECs), Spleen Sinusoidal Endothelial Cells (SSECs) and Anterior Pituitary Sinusoidal Endothelial Cells (APSECs). While SECs share many functional characteristics with other endothelial sub types (high endocytic capacity, responsiveness to shear-stress and inflammation), they also have unique sinusoidal characteristics which distinguish them from large vessel endothelium and other microvascular beds (high scavenging capacity (CD32B, STAB2, LYVE1, CD14, MRC1 mediated), and fenestration in dynamically responsive sieve plate architectures) (Braet and Wisse, 2002; Deleve, 2013a; Elvevold et al., 2008).

The functional differences between types of endothelial cells are associated with both developmentally controlled specification mechanisms (e.g. artery, vein, endocardium, lymphatic specification) and anatomically/tissue associated specification as in capillary types: continuous (brain, lung, skeletal muscle), fenestrated (kidney, intestine, choroid plexus), sinusoidal (liver, bone marrow, spleen, anterior pituitary). While the anatomical and liver tissue associated signaling is incompletely understood, Martinez has demonstrated the influence of oxygen tension on the structure and function of isolated liver sinusoidal endothelial cells (Martinez et al., 2008).

(Ditadi et al., 2015) (Zhang et al., 2017), has shown that VEGF, bFGF, NOTCH and signaling play a role in the specification of vascular fates in hPSC differentiation cultures.

### SUMMARY

In one aspect, methods of producing sinusoidal endothelial cell-like cells (SEC-LCs) are provided. Such methods typically include providing stem cells or angioblasts; and culturing the stem cells or angioblasts under conditions in which SEC-LCs are produced, wherein the conditions comprise: a) culturing the stem cells or angioblasts in the presence of bFGF; or b) culturing the stem cells or angioblasts in the presence of vascular endothelial growth factor (VEGF)-A to produce endothelial cells followed by culturing the endothelial cells in the presence of a TGF-beta signaling inhibitor, cyclic AMP (cAMP) signaling agonist, VEGF-C; or c) culturing the stem cells or angioblasts under hypoxic conditions, thereby producing SEC-LCs.

In some embodiments, the SEC-LCs are liver SEC-LCs. In some embodiments, the stem cells are pluripotent stem cells, induced pluripotent stem cells, or embryoid bodies. The angioblasts can be venous angioblasts or arterial angioblasts. In some embodiments, the stem cells are further cultured in the presence of BMP4, bFGF, and/or CHIR. In some embodiments, the stem cells are cultured in the presence of Notch inhibitor or a MEK inhibitor, bFGF and/or a venous angioblast specifying concentration of VEGF (a venous angioblast inducing media). In some embodiments, the method further includes culturing the stem cells or angioblasts in the presence of a Notch inhibitor (e.g., GSI). In some embodiments, the SEC-LCs are cultured in the presence of TGFbeta signaling inhibitor, a cAMP signaling agonist, and/or a deficiency in VEGF-C.

In some embodiments, the cAMP signaling agonist is cAMP, 8-Br-cAMP, forskolin and/or IBMX. In some embodiments, the TGFbeta signaling inhibitor is SB431542. In some embodiments, the hypoxic conditions comprise culturing in the presence of 5% CO₂/5% O₂ or culturing in the presence of a hypoxia inducible factor (HIF) prolyl-hydroxylase (PHD) inhibitor (HIF-PHDI). Representative HIF-PHDIs include, without limitation, tricyclic triazole compounds (e.g., IOX2, IOX4, or DMOG). Additional representative HIF-PHDIs include, without limitation, Daprodustat, Molidustat, Roxadustat, Vadadustat or Desidustat.

In some embodiments, the SEC-LCs express Factor VIII. In some embodiments, the method further includes monitoring the SEC-LCs for the presence of Factor VIII. In some embodiments, the method further includes isolating the SEC-LCs.

In another aspect, populations of SEC-LC cells are provided that are produced by the methods described herein.

In still another aspect, methods of screening test compounds for binding and uptake by liver SEC-LCs are provided. Such methods typically include contacting SEC-LCs made as described herein with a test compound, and determining whether or not the SEC-LCs bind and uptake (e.g., scavenge) the test compound (e.g., on the cell surface or intracellularly). In some embodiments, such methods further include determining the rate of clearance or scavenge.

In some embodiments, the test compound is a therapeutic antibody. Representative test compounds include, without limitation, monoclonal antibodies, cytokines, or small molecules.

In yet another aspect, methods of treating an individual suffering from a liver disease are provided. Such methods typically include introducing SEC-LCs made as described herein into the individual. In some embodiments, the administering is directly to the liver. In some embodiments, the administering is systemic.

Representative liver diseases include, without limitation, nonalcoholic steatohepatitis (fatty liver disease or NASH), progressive cirrhosis diseases or disorders, Hemophilia A, or hepatocellular carcinoma (HCC).

An aspect of the present disclosure is directed to a method of producing a population of venous angioblast cells comprising culturing KDR+CD56+CD34− mesoderm in a venous angioblast media comprising a Notch inhibitor or a MEK inhibitor, a venous angioblast specifying concentration of VEGF and/or bFGF, until a cell population comprising CD34+ CD73+CD184− venous angioblast cells are obtained; and optionally isolating a CD34+ population from the cell population comprising CD34+ CD73+CD184− venous angioblast cells, optionally using a CD34 affinity reagent, CD31 affinity reagent and/or CD144 affinity reageant, optionally alone or in combination with a CD73 affinity reagent.

In one embodiment, the method is for producing a population of venous endothelial cells the method comprising preparing a population of venous angioblast cells as described herein and culturing the isolated CD34+ population in venous endothelial inducing media to produce venous endothelial cells, optionally wherein the venous endothelial cells comprise PDGFRβ−, CD73+ venous endothelial cells

A further aspect includes a method of producing sinusoidal endothelial cell like cells (SEC-LCs) comprising obtaining a population comprising CD34+CD73+CD184− venous angioblast cells, optionally wherein the CD34+CD73+CD184− venous angioblast cells comprise at least 50%, at least 60%, at least 70%, at least 80% or at least 90% of the population; and differentiating the CD34+CD73+CD184− venous angioblast cells in vitro to obtain SEC-LCs.

Also provides are cells produced according to a method described herein, compositions and kits comprising said cells and/or components for making said cells and uses thereof.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### DRAWINGS

An embodiment of the present disclosure will now be described in relation to the drawings in which: FIG. 1: Mesodermal hPSC Differentiation to Angioblasts by Modulation of VEGFA and bFGF. (1A) Schematic of EB-based hPSC differentiation to venous angioblasts. (1B) Heatmap comparing kinetic gene expression profiles of day 0-8 bulk EB populations and day 8 CD34+ MACS enriched angioblasts following venous differentiation (day 4-8 10 ng/ml VEGFA, 30 ng/ml bFGF 10 μm GSI) as in (1A). High (dark) and low (light) expression levels are indicated for each gene. (1C) Representative flow cytometric analysis of day 4 KDR+CD56+CD235a-mesodermal cells and day 8 CD34+CD31 low venous angioblasts before and after MACS enrichment. (1D) Representative flow cytometric analysis and quantification of day 6 CD34+ angioblasts specified under different day 4-6 treatment conditions of VEGFA (0-100 ng/ml) and bFGF (0-100 ng/ml) including bias of CD34+ cells to CD184+CD73 low arterial or CD184-CD73 high venous angioblasts (ANOVA with Bonferroni test, *p<0.05, **p<0.01, ***p<0.001 between VEGFA and bFGF doses as indicated and between identified CD34+ subpopulations). For all qRT-PCR analysis, expression values are normalized to levels of the housekeeping gene TBP. Error bars represent SEM.

FIG. 2: Specification of Arterial and Venous Angioblasts. (2A) Schematic of EB-based hPSC-derived day 4 mesoderm differentiation to venous or arterial angioblasts (day 4-8) and subsequent monolayer (day 8-12) endothelial cells. (2B-2H) Representative flow cytometric analysis and quantification of day 8 arterial (day 4-8: 100 ng/ml VEGFA; V100) and venous (day 4-8: 10 ng/ml VEGFA and 10 μM GSI; V10G) angioblasts pre and post MACS purification and outgrowth of endothelial cells from purified angioblasts (ANOVA with Bonferroni test, *p<0.05, **p<0.01, ***p<0.001 between CD34+ populations as indicated and between CD34+ subpopulations). (20 Flow cytometric analysis of CD73 mean fluorescence intensity (MFI) of day 12 arterial and venous endothelial cells (total CD34+) (ANOVA with Bonferroni test, *p<0.05 as indicated). (2J-2L) Heatmaps comparing day 8 and day 12 purified CD34+ or CD34+CD31+ cells for expression of markers of arterial, venous and general endothelial fate (2J), NOTCH signalling (2K), and lymphatic and LSEC markers (2L) (ANOVA with Bonferroni test, *p<0.05 between arterial and venous population at given day for a given gene). For all qRT-PCR analysis, expression values are normalized to levels of the housekeeping gene TBP. Error bars represent SEM.

FIG. 3: Specification of LSEC-LCs from Venous Angioblasts. (3A) Schematic of EB-based hPSC-derived day 4 mesoderm differentiation to venous angioblasts, endothelial cells and LSEC-like cells (LSEC-LCs). (3B) Heatmaps comparing gene expression relative to TBP control gene of LSEC marker and related genes in day 16 cultures after 4 days of treatment with different concentrations of 8-Br-cAMP (cAMP; 0, 0.1, 0.3, 1.0 mM), SB-431542 (SB; 0, 6, 18 μM), and VEGFC (0, 100 ng/ml). Each heatmap scale is relative to the indicated gene (ANOVA with Bonferroni test, *p<0.05, **p<0.01, ***p<0.001 indicated condition vs 0 mM cAMP at given SB/VEGFC dose, +p<0.05, ++p<0.01, +++p<0.001 indicated condition vs 0μM SB /0 ng/ml VEGFC at given cAMP dose). (3C) Fluorescence-activated cell sorting (FACS)-mediated purification of CD31+ cells and qRT-PCR analysis of LSEC markers and endothelial genes expressed in day 16 venous LSEC-LCs after 1 mM cAMP 6 μM SB treatment from day 12-16 (ANOVA with Bonferroni test, *p<0.05, **p<0.01, ***p<0.001 as indicated). For all qRT-PCR analysis, expression values are normalized to levels of the housekeeping gene TBP. Error bars represent SEM.

FIG. 4: Specification of LSEC-LCs from Arterial Angioblasts. (4A) Schematic of EB-based hPSC-derived day 4 mesoderm differentiation to arterial angioblasts, endothelial cells and LSEC-like cells (LSEC-LCs). (4B) Heatmaps comparing gene expression relative to TBP control gene of LSEC marker and related genes in day 18-20 cultures after 6-8 days of treatment with different concentrations of 8-Br-cAMP (cAMP; 0, 0.1, 0.3, 1.0 mM), SB-431542 (SB; 0, 6, 18 μM), and VEGFC (0, 100 ng/ml). Each heatmap scale is relative to the indicated gene (ANOVA with Bonferroni test, *p<0.05, **p<0.01, ***p<0.001 indicated condition vs 0 mM cAMP at given SB/VEGFC dose, +p<0.05, ++p<0.01, +++p<0.001 indicated condition vs 0μM SB/0 ng/ml VEGFC at given cAMP dose). (4C) Fluorescence-activated cell sorting (FACS)-mediated purification of CD31+/LYVE1+ cells from indicted populations (Pop.) in day 18-20 arterial LSEC-LCs after 1 mM cAMP, 6 μM SB treatment from day 12-20. qRT-PCR analysis of given populations (ANOVA with Bonferroni test, *p<0.05, comparing indicated population to population 8). For all qRT-PCR analysis, expression values are normalized to levels of the housekeeping gene TBP. Error bars represent SEM.

FIG. 5: LSEC-LC Specification is Enhanced in Venous Angioblasts in Hypoxia. (5A) Schematic of EB-based hPSC-derived day 4 mesoderm differentiation to arterial and venous angioblasts, endothelial cells and LSEC-like cells (LSEC-LCs) under standard LSEC induction conditions (6 μM SB, 1 mM cAMP, 30 ng/ml bFGF) and defined oxygen conditions. (5B-5C) Representative flow cytometric analysis and quantification of arterial and venous CD31 and LYVE1 expression profiles during LSEC-LC induction (day 12-20) under hypoxic (5% O₂) and normoxic (21% O₂) conditions (ANOVA with Bonferroni test, *p<0.05, comparing indicated days to arterial hypoxic day 12). (5D-5E) Representative flow cytometric analysis and quantification of arterial and venous CD31+ cells for expression of LYVE1 and CD32 during LSEC-LC induction under hypoxic and normoxic conditions (ANOVA with Bonferroni test, *p<0.05, comparing indicated days to day 12 of given oxygen tension and cell type, #p<0.05, comparing hypoxia cultured artery and venous cells at indicated days to corresponding day, $p<0.05, comparing hypoxia and normoxia at indicated days to the corresponding day within the cell type). (5F-5H) qRT-PCR analysis of hypoxic and normoxic cultured arterial and venous LSEC-LC cultures for LSEC markers (ANOVA with Bonferroni test, *p<0.05, comparing hypoxia and normoxia at indicated days to the corresponding day within the cell type, #p<0.05, comparing hypoxia cultured arterial and venous cells at indicated days to corresponding day). (5I) qRT-PCR analysis of hypoxic cultured LSEC-LCs induced from arterial and venous endothelial cells purified by FACS for CD31+ cells at day 14-16 (ANOVA with Bonferroni test, *p<0.05, comparing CD31+ and CD31− within indicated cell type, #p<0.05, comparing CD31+ between artery and venous origin cell types). For all qRT-PCR analysis, expression values are normalized to levels of the housekeeping gene TBP. Error bars represent SEM.

FIG. 6: CD32B Expression is Regulated by Hypoxia/HIFla Signalling. (6A) Schematic of EB-based hPSC-derived day 4 mesoderm differentiation to arterial and venous angioblasts, endothelial cells and LSEC-like cells (LSEC-LCs) under standard LSEC induction conditions (6 μM SB, 1 mM cAMP, 30 ng/ml bFGF) and defined oxygen conditions with the addition of IOX2. (6B) Representative flow cytometric analysis of normoxic cultured CD31+ arterial LSEC-LCs treated with DMSO or IOX2 at increasing doses (days 12-16). (6C) qRT-PCR analysis of LSEC markers in day 16 arterial and venous LSEC-LCs cultured under normoxia conditions treated with DMSO or increasing IOX2 doses for 4 days (ANOVA with Bonferroni test, *p<0.05, comparing arterial and venous cells at give IOX2 doses). (6D) Representative flow cytometric analysis of day 16 arterial and venous CD31+ LSEC-LCs cultured under normoxic and hypoxic conditions with and without IOX2. For all qRT-PCR analysis, expression values are normalized to levels of the housekeeping gene TBP. Error bars represent SEM.

FIG. 7: hPSC-Derived Angioblasts Engraft the Neonatal Liver and Mature to Express Human LSEC Markers. (7A) Schematic of neonatal intrahepatic transplantation model, isolation and purification of hPSC-derived LSECs derived from CD34+ day 8 angioblasts or CD31+ day 14-16 LSEC-LCs. (7B) Engraftment of tdRFP (RFP) positive hESC-derived cells within the isolatable non-parenchymal-cell (NPC) fraction over time from individual mice with indicated transplanted populations. Effective engraftment (>1% RFP) frequency is depicted right of the legend with relation to elevated circulating FVIII levels seen in FIG. 12B. (7C-7F)

Representative histological analysis of endothelial engrafted region of NSG mouse liver derived from day 8 venous angioblast cells matured for 77 days in vivo. Human specific nuclear and LSEC marker stains are as indicated. 60 μm scale bar applies to all images. (7G) Representative flow cytometric analysis and quantification of NPC samples from transplanted arterial or venous angioblasts or arterial or venous LSEC-LCs (ANOVA with Bonferroni test, *p<0.05, between CD31+ fractions as indicated and between CD32+/LYVE1+ fractions as indicated). (7H) qRT-PCR of analysis of LSEC markers in FACS isolated fractions of invivo matured day 8 venous angioblast derived grafts. Comparison of RFP+ cells either CD31−, CD31+CD32−LYVE1−, CD31+CD32+LYVE1−, CD31+CD32+LYVE1+ to primary human LSECS isolated by FACS (Live, CD45−, CD68−, CD31+, CD32+) (ANOVA with Bonferroni test, *p<0.05, between RFP+ CD31− cells and indicated samples or additionally as indicated between RFP+CD31+ fractions). Primary human LSEC expression levels are indicated below the gene name where available. For all qRT-PCR analysis, expression values are normalized to levels of the housekeeping gene TBP. Error bars represent SEM.

FIG. 8: Replication of the LSEC Program with H1-GFP hESCs. (8A) Representative flow cytometric analysis of day 4 KDR+CD56+CD235a low-mesodermal cells after induction similarly to FIG. 1A. (8B) Representative flow cytometric analysis of day 8 CD34+CD31 low arterial and venous angioblasts after CD34 MACS enrichment including arterial (CD184+CD73low) and venous (CD184-CD73high) specification in response to 100 ng/ml VEGFA (arterial) and 10 ng/ml VEGFA and 10 μM GSI (venous) conditions between day 4 and 8. (8C) Representative flow cytometric analysis of CD31+ arterial and venous LSEC-LC induction to expression of CD32 within LYVE1+ cells in response to 6 μM SB and 1 mM cAMP addition at day 12. (8D) Schematic of NPC cell recovery from day 8 venous transplanted mice to isolate, quantify, and recover GFP+ hESC-derived LSECs. (8E-8H) Representative flow cytometric analysis of GFP+ cell recovery, GFP+% quantitation, CD31+% quantitation of GFP+ fraction, LYVE1+/CD32+ quantitation of GFP+CD31+ cells. (80 Representative scanning electron microscopic images of FACS purified, overnight cultured, fixed and processed cells from indicated sorted populations imaged at 50,000× magnification to visualize ˜100 nm fenestrations if present. Imaging conditions and scale bar are indicated on each image. (8J) Comparison of GFP+ cells either CD31−, CD31+CD32−LYVE1−, CD31+CD32+LYVE1−, CD31+CD32+LYVE1+ to primary human LSECS isolated by FACS as (Live, CD45−, CD68−, CD31+, CD32+) (ANOVA with Bonferroni test, *p<0.05, between GFP+CD31− cells and indicated samples or additionally as indicated between GFP+CD31+ fractions). Primary human LSEC expression levels are indicated below the gene name where available. For all qRT-PCR analysis, expression values are normalized to levels of the housekeeping gene TBP. Error bars represent SEM.

FIG. 9: hPSC-Angioblast-Derived Cells are Predominantly Human Zone 2/3 LSECs After In Vivo Maturation. Day 8 venous angioblast cells were transplanted intrahepatically in a neonatal NSG mouse and matured for 77 days before isolation of unfractionated DAPI− RFP+ cells that were processed for single-cell RNA-seq. (9A) After mitochondrial exclusion of dying cells, doublet exclusion, normalizaiton and standard processing, 5951 were segmented into 5 clusters based on their expression profiles (clusters 0, 1, 2, 3, 4). (9B) Expression patterns of candidate genes associated with LSECs and related cells depicting high expressing cells (darkest grey), middle expressing cells, low expressing, and undetectable expression (lightest grey) are shown. (9C) Dominant expression patterns within clusters 0-4 show elevated (light color) LSEC marker expression in clusters 0, 1, and 3 with fibroblast marker expression in clusters 2 and 4. (9D) Correlation analysis using 200 hepatic endothelium defining genes (50 most distinguishing each from MacParland's 3 endothelial clusters and 1 fibroblast/stellate cluster) were used to compare Clusters 0-4 to established primary human Liver scRNA-seq data as previously generated by MacParland et al. 2018. R² values are depicted as a heat map to allow comparison between groups with significant positive (light) or negative (dark) correlations indicated with a “*” based on an FDR-corrected p-value <0.05.

FIG. 10: scRNA-seq Selected Genes. Expression patterns of additional candidate genes associated with LSECs depicting high expressing cells (darkest grey), middle expressing cells, low expressing, and undetectable expression (lightest grey) are shown. (10A) Expression of PTPRC (CD45) is shown as a marker of hematopoietic cell types. (10B-10D) Expression of candidate fibroblast/smooth muscle/mesenchymal markers are shown. (10E) Expression based analysis of predictive cell cycle status of all cells with status depicted as indicated. (10F-10G) Expression of candidate cell cycle progression associated markers. (10H-10K) Expression of receptors and co-receptors of adrenomedulin signalling pathway. (10L-10S) Expression of established and novel LSEC markers.

FIG. 11: hPSC-Derived, In Vivo Matured LSECs are Fenestrated and Scavenge E. coli Bioparticles. (11A) Representative scanning electron microscopic images of primary human insitu vasculature imaged on the cut surface of fresh fixed liver tissue. LSECs, terminal portal venous endothelium and hepatic artery endothelium are shown for comparison of presence (indicated by white arrows) or absence of fenestrations. (11B) Representative scanning electron microscopic images of day 8 venous angioblast-derived, in vivo-matured, FACS-isolated based on indicated marker panels, overnight-cultured, treated for 30 minutes with or without fenestration (white arrows) stimulating 0.1 μg/ml Latrunculin A, fixed and processed for SEM. Imaging conditions and scale bar are indicated on each image. (11C) Quantification of fenestrated cells (>10 fenestrations arranged in a defined sieve plate cluster), endothelial cells (smooth edged cells with angiogenic lateral sprouts free from fibroblast-like adhesions), non-endothelial cells (fibroblast-like cells and rounded cells) was performed manually on the indicated numbers of cells from a total of at least 4 independent replicates pooling the data. Comparative in vitro derived cells including venous LSEC-LCs sorted as CD31+CD32+LVYE1+ on day 18, replated and cultured for an additional 10 days in addition to standard multi-donor derived HUVECs that were also quantified with and without Latrunculin A stimulation. (11D-11E) Quantification of fenestration size (longest diameter) in (11C) primary human LSECs in situ (black), venous angioblast-derived, in vivo matured and isolated LSECs with (white) and without (grey) latrunculin A stimulation. (11E) Quantification of surface area of LSECs covered by fenestrations before and after latrunculin stimulation. Error bars represent SEM. (11F-11G) Representative flow cytometric analysis of E. coli bioparticle binding in the presence of human IgG by the indicated venous angioblast-derived populations isolated at 72-99 days following neonatal transplantation (ANOVA with Bonferroni test, *p<0.05, between indicated groups). Error bars represent SEM.

FIG. 12: hPSC-Derived Angioblasts Engraft the Monocrotaline Injured Adult Liver and Mature to Express Human LSEC Markers. (12A) Schematic of monocrotaline injured (150 mg/kg; IP) adult NSG mouse intrasplenic transplantation model. Four to six weeks post transplantation, liver digestion releases non-parenchymal cell (NPC) fraction facilitating FACS-mediated isolation of hPSC-derived RFP+ cells with quantitative sub fractionation based on human LSEC markers huCD31, CD32, huLYVEl. (12B) Circulating human specific FVIII antigen in the blood of non-transplanted, neonatal intrahepatic HUVEC transplanted, neonatal intrahepatic hPSC-derived cell transplanted, and adult intrasplenic hPSC-angioblast transplanted NSG mice. Neonatal hPSC-derived cell transplanted mice were binned according to human engraftment level (RFP% of NPC fraction). Adult transplanted mice, 31-41 days post-transplant, despite the short engraftment timeline, show FVIII levels with mice in the low therapeutic range (˜2-4% normal human levels). (12C-12F) Representative histological analysis of venous angioblast engrafted adult mouse after 41 days of in vivo maturation. Human specific nuclear marker (KU80) and LSEC markers (CD31, CD32B, LYVE1, STAB2) were probed as indicated. 60 μm scale bar applies to all images. (12G) Representative flow cytometric analysis and quantification of NPC fractions from adult mice transplanted intrasplenically with day 8 venous or arterial angioblasts. (12H) Quantification of LSEC engraftment in adult transplanted mice including: engraftment efficiency (RFP% of NPC, maximal engraftment is ˜60-70% of NPC), endothelial contribution (% CD31), LSEC contribution (% CD32 and LYVE1 expression pattern). (T-test, *p<0.05, between %RFP+ as indicated).

### DESCRIPTION OF VARIOUS EMBODIMENTS

The inventors have developed methods for making angioblasts which are enriched for venous endothelial progenitors, and which can be used to generate sinusoidal endothelial cell like cells in vitro. These progenitors are able to be differentiated in vitro and in vivo and show characteristics of functional sinusoidal cells when transplanted into the liver. The inventors have found that using a specifying media low in VEGFA and/or isolating a CD34+ fraction from angioblast cells, for example, after low VEGFA treatment for 1 or more days (e.g., 2, 3, 4, 5, 6, 7, 8, 9, 10, 11 or 12 or more days), produces a population of venous angioblasts that can be differentiated to produce increased numbers of SEC-LCs and are capable of robust engraftment.

### Definitions

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. For example, the term “a cell” includes a single cell as well as a plurality or population of cells, “an agonist” inclused. Generally, nomenclatures utilized in connection with, and techniques of, cell and tissue culture, molecular biology, and protein and oligonucleotide or polynucleotide chemistry and hybridization described herein are those well-known and commonly used in the art (see, e.g. Green and Sambrook, 2012).

Terms of degree such as “about”, “substantially”, and “approximately” as used herein mean a reasonable amount of deviation of the modified term such that the end result is not significantly changed. These terms of degree should be construed as including a deviation of at least +5% of the modified term if this deviation would not negate the meaning of the word it modifies.

The term “pluripotent stem cell” as used herein refers to a cell with the capacity to differentiate into cells of the three germ cell layers. Pluripotency is also evidenced by the expression of embryonic stem (ES) cell markers (e.g., POU5F1+, SOX2+, NANOG+, SSEA3+. SSEA4+, and SSEA5+). Suitable pluripotent cells for use herein include embryonic siam cells. (ESCs; e.g., human ESCs) such as, for example, mesoderm cells (e.g., human mesoderm cells that express, for example, KDR+CD56+CD34−), induced pluripotent stem (iPS) cells (e.g., human iPS cells), or cells from embryoid bodies (e.g., cells from human embryoid bodies).

“Angioblasts” as used herein refer to cells that differentiate from the mesoderm that are the progenitor cells from which blood vessels arise. Angioblasts can be identified by the following markers (or the absence thereof): CD34+, KDR+, CD31−/low, and CD144−/low. For example, venous angioblasts can be identified as CD34+ CD73+ CD184−, while arterial angioblasts can be identified as CD34+ CD73low CD184+.

Typically, cells having a venous lineage are identified by the expression of NRP2, ALPNR, CD73, EPHB4, and NR2F2, whereas cells having an arterial lineage are identified by the expression of EFNB2, CXCR4, NRP1 and NRP2.

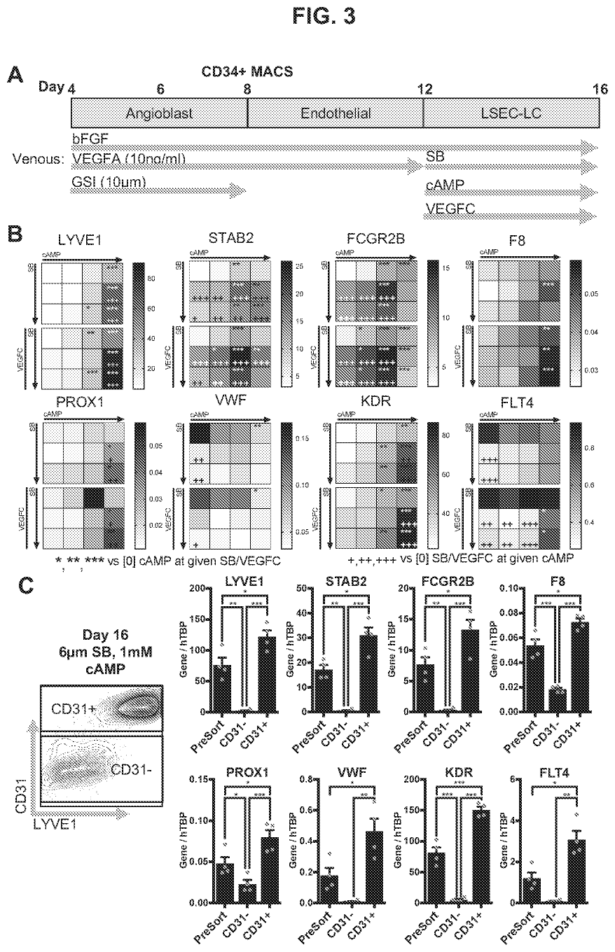

The term “sinusoidal endothelial cell like cells (SEC-LCs)” as used herein refer to an in vitro produced cell or population that are produced using a method described herein, correspond to day 13 or later cells as described in FIG. 3A and which express, for example, one or more of CD31, LYVE1, FCGR2B/CD32B, STAB2, PLVAP, F8, CD36 and/or GATA4. They can also refer to the in vitro produced cells after they are introduced into a subject.

Liver SECs (LSECs) can be identified through their dynamically controlled transcellular fenestrations arranged in sieve plates (Wisse et al., 1985). Other types of Sinusoidal Endothelial Cells (SECs) exist in addition to LSECs including Bone Marrow Sinusoidal Endothelial Cells (BSECs), Spleen Sinusoidal Endothelial Cells (SSECs) and Anterior Pituitary Sinusoidal Endothelial Cells (APSECs) that are defined by their expression of general endothelial markers (CD31, CD144), the presence of transcellular fenestrations that lack a restrictive diaphragm, and are dialated with a 30-40 μm crosssection. Anatomically, these SEC subtypes are primarily defined by the organs from which they are derived.

The term “venous angioblast inducing media” as used herein refers to a media suitable for inducing mesodermal cells to differentiate into venous angioblasts. Venous angioblast inducing media comprises a base media and one or more venous specifying components, such as a Notch inhibitor and/or a MEK inhibitor, bFGF (or equivalent FGF) and/or a venous angioblast specifying concentration of VEGFA. For example, the base media can for example be commercially available StemPro34 (ThermoFisher Scientific, 10639011) used as supplied or partially diluted with IMDM (ThermoFisher Scientific, 12200036) further supplemented with ITS-X (ThermoFisher Scientific, 51500056) additional glutamine, ascorbic acid, monothioglycerol and transferrin. As described in Example 1, culturing mesoderm in the presence bFGF at a concentration of 5 ng/mL and VEGFA at concentrations between 0 and 30 ng/mL, preferably between 0 and 20 ng/mL, produced an increased number of venous angioblasts. Other base medias such as GMEM, DMEM, and RPMI optionally comprising other supplements can also be used. The optimal concentration of a component such as VEGFA and/or FGF can be determined empirically and depends for example on the concentration of endogenously produced component. VEGFA and FGF can be endogenously produced. Adding exogenous VEGFA can increase the yield of CD34+. Adding exognenous FGF can supplement any endogenous FGF produced and sustain cell numbers. For example, the concentration of 5 to 30 ng/ml can be used. Elevated FGF levels are mostly to sustain angioblasts to day 8 and to keep all the cells growing (ie maintining total cell numbers). The venous angioblast inducing media may also include one or more of a MEK inhibitor, a NOTCH agonist such as resveratrol), a PI3K inhibitor for example at concentrations used in Ditadi et al, 2015, BMP or a BMP agonist, insulin, a TGFbeta signaling inhibitor, PDGF-BB, LDL, L690 (Impase inhibitor), inositol, Resveratrol (NOTCH agonist), as in Zhang et al, 2017.

The term “venous angioblast specifying concentration of VEGFA” as used herein means a concentration of VEGFA that promotes development of venous angioblasts when applied to mesoderm, for example prepared as described herein. This can for example be any concentration from about 0 to about 30 ng/mL of VEGFA, for example from about 0 to about 20 ng/mL with the most suitable concentration determined in titration experiments for example similar to those described in Example 1.

The term “inhibitor” as used herein refers to a chemical compound, drug, biomolecule, or composition or mixture thereof that is capable of inhibiting the identified activity. For example, the term “Notch inhibitor” as used herein means any such moiety that is capable of inhibiting Notch receptor ligand binding, Notch extracellular domain cleavage, Notch intracellular cleavage after endocytosis by gamma secretase, etc., that results in a reduction of Notch activity, measured, for example, by detecting transcription factor CSL levels, and includes for example gamma secretase inhibitors (GSIs) such as L-685,458 (Tocris, Bristol UK). In addition, the term “TGFbeta signaling inhibitor” as used herein means any such moiety that is capable of inhibiting functional activation of TGFbeta signaling (e.g., via inhibition of TGFbeta ligand secretion), sequestration of released TGFbeta ligand from solution (e.g., via a TGFbeta binding antibody),

TGFbeta receptor (e.g., via a blocking antibody or small molecule that prevents receptor activation), receptor activation of downstreatm signaling (e.g., via blockade of kinase signing to activate SMAD substrates (e.g., the mechanism of action of SB-431542)). An inhibitor may result in a reduction of at least 50%, at least 60%, at least 70%, at least 80%, or at least 90%, relative to a sample untreated with the inhibitor.

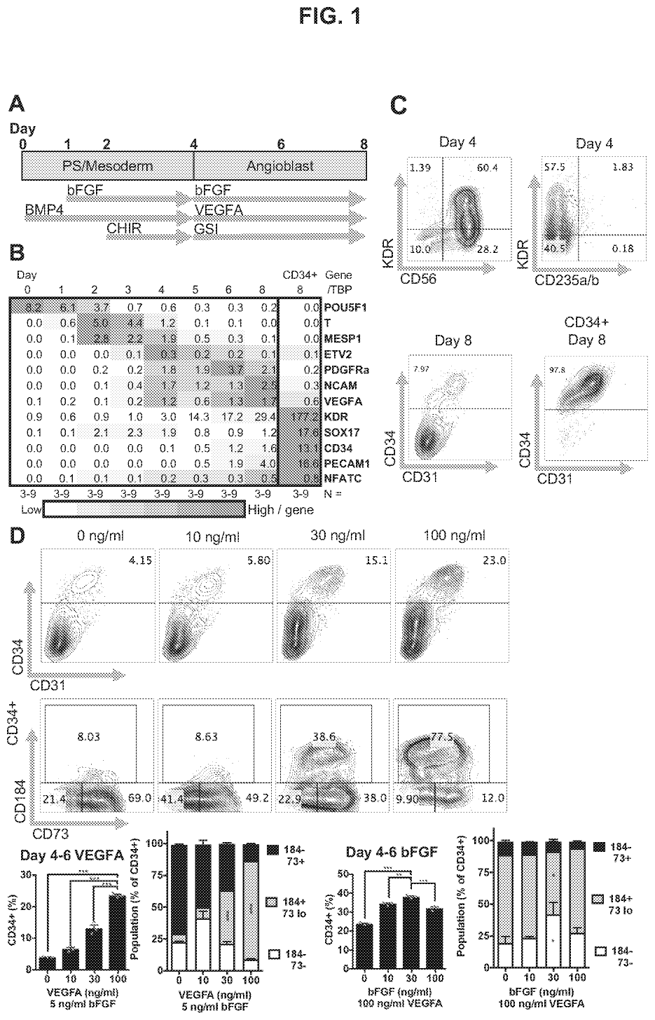

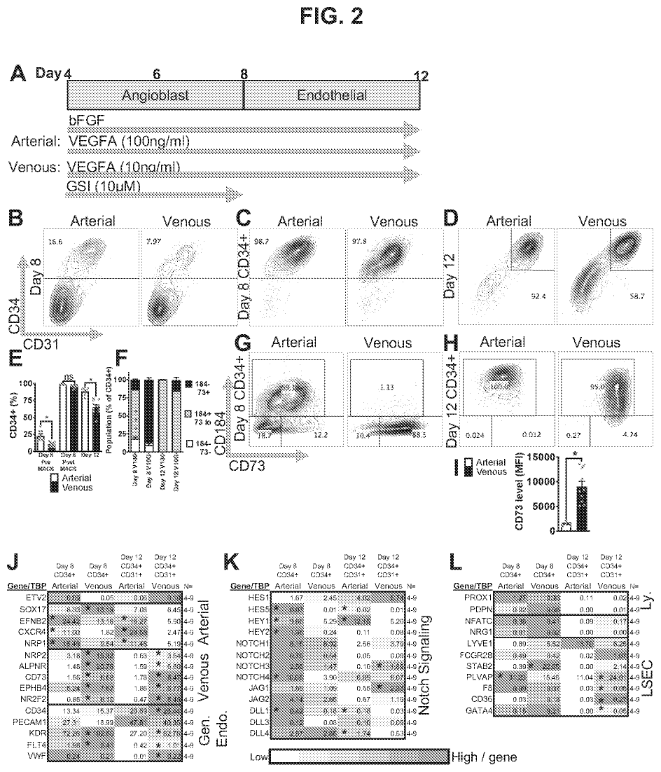

The term “venous endothelial inducing media” as used herein refers to a media suitable for inducing angioblasts to differentiate into endothelial lineage cells. Venous endothelial inducing media comprises a base media and one or more endothelial specifying components such as bFGF (or equivalent FGF) and/or an endothelial specifying concentration of VEGFA. For example, the base media can for example be commercially available StemPro34 (ThermoFisher Scientific, 10639011) used as supplied or partially diluted with IMDM (ThermoFisher Scientific, 12200036) further supplemented with ITS-X (ThermoFisher Scientific, 51500056) additional glutamine, ascorbic acid, monothioglycerol and transferrin. As described in Example 1, culturing CD34 enriched day 8 angioblasts with VEGFA at a concentration of 10 ng/mL produced a population of cells expressing increased levels of venous genes (e.g. NRP2, ALPNR, CD73, EPHB4, NR2F2) and lower levels of arterial genes (e.g. EFNB2, CXCR4, NRP1 AND NRP2) compared to angioblasts cultured in the presence of 100 ng/ml of VEGF- as shown in FIG. 2J. Other base medias such as GMEM, DMEM, and RPMI optionally comprising other supplements can also be used to expand venous endothelial cells. Said media which is used for example for days 8-12 in the method shown in FIG. 2A, lacks GSI or other Notch inhibitor.

The term “venous endothelial specifying concentration of VEGFA” as used herein means a concentration of VEGFA that promotes development and proliferation without loss of venous marker expression of venous endothelial cells. This can for example be any concentration from about 0 to about 30 ng/mL of VEGFA, for example from about 0 to about 20 ng/mL with the most suitable concentration determined in titration experiments for example similar to those described in Example 1.

The term “SEC inducing media” as used herein refers to a media suitable for inducing precursor endothelial lineage cells to differentiate to sinusoidal endothelial cells and/or sinusoidal endothelial cell like cells (SEC-LCs). SEC inducing media comprises a base media and one one or more SEC specifying components and/or endothelial sustaining components, such as bFGF (or equivalent FGF) (e.g., 0-30 ng/ml) and/or VEGFA (e.g. 0-30 ng/ml, preferably 0-20 ng/mL), for example comprising 30 ng/ml bFGF and 0 ng/ml VEGFA. VEGFA can be included or excluded, with concentrations included only to an endothelial specifying concentration. The SEC specifying components can comprise a TGFbeta signaling inhibitor and cAMP signaling agonist.

For example, the base media can for example be commercially available StemPro34 (ThermoFisher Scientific, 10639011) used as supplied or partially diluted with IMDM (ThermoFisher Scientific, 12200036) further supplemented with ITS-X (ThermoFisher Scientific, 51500056) additional glutamine, ascorbic acid, monothioglycerol and transferrin. Other base medias such as GMEM, DMEM, and RPMI optionally comprising other supplements can also be used.

The term “cAMP agonist” or “cAMP signaling agonist” as used herein means any cAMP analog and/or activator (e.g. signaling agonist or cAMP degradation inhibitor), including cAMP. Exemplary cAMP analogs include, but are not limited to, dibutyryl cAMP, 8-Bromo-cAMP, and Sp-8-Br-cAMPS (8-bromoadenosine-3′,5′-cyclicmonophosphorothioate, Sp-isomer) and combinations thereof. Exemplary cAMP activators include forskolin, IBMX (and other xanthine derivatives that increase cAMP) and rolipram and the like as well as combinations thereof.

The term “LSEC markers” and “SEC markers” can be used interchangeably as LSEC markers are SEC markers that are detected in liver derived SECs. Although different SEC types may express specific genes in the context of a specific organ, in vitro, prior to implantation for example, these markers can be used to identify sinusoidal endothelial lineage cells. SEC markers can include markers identified herein as well as combination thereof or other markers associated with SEC.

As used herein, the term “pharmaceutically acceptable carrier” is intended to include any and all solvents, dispersion media, coatings, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Optional examples of such carriers or diluents include, but are not limited to, water, saline, ringer's solutions, dextrose solution, and 5% human serum albumin and bovine serum albumin (BSA).

### Methods and Compositions

Provided herein are methods for making angioblasts enriched for venous angioblast cells as well as isolated populations thereof.

Accordingly one aspect includes a method of producing a population comprising enriched for venous angioblast cells, the method comprising: culturing KDR+CD56+CD34− mesoderm in a venous angioblast media comprising a venous angioblast specifying concentration of VEGFA, until a cell population comprising CD34+ CD73+CD184− venous angioblast cells are obtained.

For example, in one embodiment, the method of producing a population of angioblast cells with enhanced ability to form sinuisoidal endothelial cells (e.g. liver sinusoidal endothelial cells) in vitro, and engraft more robustly in vivo, said method comprising culturing KDR+CD56+CD34− mesoderm in an enrichment media (e.g. venous angioblast media) comprising a Notch inhibitor and/or a MEK inhibitor, and an effective amount of VEGFA and/or bFGF sufficient to generate a population of angioblasts enriched for venous angioblasts as compared with arterial angioblasts, wherein said venous angioblasts are those cells which are CD34+CD73+CD184−. Said population of venous angioblast cells thus generated is, for example, capable of generating sinusoidal endothelial cells in vitro in the presence of a TGFbeta signaling inhibited environment, and can for example have an increased capability to generate sinusoidal endothelial cell- like cells (SEC-LCs) in vitro and/or are capable of engrafting more robustly in vivo. The increased capability to generate SEC-LCs may be relative to, for example a population having a greater proportion of arterial angioblasts, such as one obtained using an increased amount of VEGFA and/or unsorted angioblasts as shown in the Examples. The venous angioblast media can be any media that comprises angioblast specifying components wherein the VEGFA is provided at a venous angioblast specifying concentration.

In one embodiment, the venous angioblast specifying concentration of VEGFA is a concentration of from about 0 to about 30 ng/mL, optionally from about 0-20 ng/mL or any 0.1 increment from 0.1 to 29.9 ng/mL. In another embodiment, the venous angioblast specifying concentration of VEGFA is 0 ng/mL meaning that no VEGFA is provided.

In one embodiment, the cells subjected to specification have less than 50%, less than 40%, less than 30%, less than 20% or less than 10% arterial lineage cells in sorted CD34+ populations, based for example on the markers described herein.

If arterial angioblasts are desired, a higher concentration of VEGFA can be used, for example greater than 30 ng/mL and for example up to 200 ng/mL, preferably 100 ng/mL.

A suitable concentration of VEGFA can be determined by performing one or more titrations similar to the titration of 0 to 100 ng/mL described in Example 1.

The venous angioblast media comprises one or more angioblast specifying components. Angioblast specifying components can include for example a FGF component such as bFGF and a Notch inhibitor such as a gamma secretase inhibitor (GSI) or a MEK inhibitor such as PD0325901. As shown in Examples 1, 3 and 4, the venous angioblast media can comprise bFGF and a GSI in addition to the venous specifying concentration of VEGFA.

The FGF component is preferably bFGF (also referred to as FGF2) but can be any FGF or FGF analog that promotes angioblast specification. The FGF component when bFGF, can be provided at a concentration from about 10 ng/ml to about 100 ng/ml, for example from about 0.1 ng/mL to about 100 ng/mL or any 0.1 increment from 0.1 to 99.9 ng/mL, preferably from about 1 ng/mL to about 30 ng/mL, optionally at about 5 ng/mL.

The Notch inhibitor can be a GSI. The GSI can be, for example, L-685,458 provided, for example, at a concentration of from about 0.1 microM to about 100 microM, or any 0.1 increment from and including 0.1 microM to 100 microM, for example, less than about 30 microM, optionally, about 10 microM. GSI is a small molecule that disrupts NOTCH signaling in vitro. GSI blocks an enzyme that cleaves the NOTCH receptor that naturally mediates NOTCH signaling. The purpose of disrupting the NOTCH pathway is to enhance the proper specification of a venous angioblast, which is then turned into a venous endothelial cell that eventually becomes a venous endothelial derived LSEC-LC. If a NOTCH inhibitor is not used, more artery angioblasts are produced, which, as described herein, are less efficient at becoming LSEC-LCs.

Other angioblast specifying components and concentrations can be used. For example, a MEK inhibitor can be used in addition to or instead of a Notch inhibitor. Suitable angioblast specifying components include for example TGFbeta signaling inhibitor.

Exemplary TGFbeta signaling inhibitors include SB431542 [4-(4-(benzo[d][1,3]dioxol-5-yl)-5-(pyridin-2-yl)-1H-imidazol-2-yl)benzamide] (Tocris, Bristol UK), and/or A83-01 [3-(6-Methyl-2-pyridinyl)-N-phenyl-4-(4-quinolinyl)-1H-pyrazole-1-carboth-ioamide] (Tocris, Bristol UK), optionally provided at a concentration range from about 1 to about 50 microM, preferably between 2 and 40 microM and even more preferably, between 2 and 10 microM.

Other TGFbeta signaling inhibitors are known in the art and are commercially available. Examples include E-616452 [2-(3-(6-Methylpyridin-2-yl)-1H-pyrazol-4-yl)-1,5-naphthyridine]; SB 505124 [2-[4-(1,3-Benzodioxol-5-yl)-2-(1,1-dimethylethyl)-1H-imidazol-5-yl]-6-methyl-pyridine];GW 788388 [4-[4-[3-(2-Pyridinyl)-1H-pyrazol-4-yl]-2-pyridinyl]-N-(tetrahydro-2H-pyran-4-yl)-benzamide]; and SB 525334 [6-[2-(1,1-Dimethylethyl)-5-(6-methyl-2-pyridinyl)-1H-imidazol-4-yl]quinoxaline].

Other factors that can be added and that have been used to influence arterial and venous specification include for example a MEK inhibitor, a NOTCH agonist, a PI3Kinhibitor (e.g. Ditadi et al 2015) and/or BMP, Insulin, TGFbeta signaling inhibitor, PDGF-BB, LDL, L690 (Impase inhibitor), Inositol, Resveratrol (NOTCH agonist) (e.g. Zhang et al, 2017)

For example, base medias and/or components described in Ditadi et al 2015 and Zhang et al 2017 can also be used whether embryoid body, monolayer or combinaiton differentiation methods are preferred.

Exemplary medias comprising suitable components are provided in Example 3 and 4. In one embodiment, the KDR+CD56+CD34− mesoderm is cultured in venous angioblast media until the cell population comprises at least or about 5%, at least or about 6%, at least or about 7%, at least or about 8%, at least or about 9%, at least or about 10% at least or about 11%, at least or about 12%, at least or about 13%, at least or about 14%, at least or about 15%, at least or about 16%, at least or about 17%, at least or about 18%, at least or about 19%, or at least or about 20%, or more CD34+CD73+CD184− venous angioblast cells for example prior to sorting or selective proliferation methods, and for example at least or about 50%, at least or about 60%, at least or about 70% or at least or about 80%, at least or about 85%, at least or about 90% or more CD34+CD73+ venous endothelial cells post sorting and/or selective proliferation. For example, using methods described herein, it is possible to obtain approximately 10-20% venous angioblasts when cells are cultured in EBs and higher, for example greater than 20%, in monolayer systems.

In one embodiment, the culturing in the venous angioblast inducing media is for at least about 1 day, optionally up to about 30 days (e.g., 1 to 18 days, 1 to 8 days, or any number of days between and/or including 1 and 30 days). For example, the culturing may be about 1 day, about 2 days, about 3 days, about 4 days, about 5 days, about 6 days, or about 10 days.

Referring to FIG. 1A, culturing KDR+CD56+CD34− mesoderm in a venous angioblast media comprising a venous angioblast specifying concentration of VEGFA, until a cell population comprising CD34+CD73+CD184− venous angioblast cells are obtained corresponds to days 4 to 8. As seen in FIG. 1B, said cells begin to express CD34 and endothelial progenitor marker ETV2 and decreased levels of Pou5F1. The expression of one or more of these genes can be used to monitor for the emergence of the CD34+CD73+CD184− population.

The KDR+CD56+CD34− mesoderm can be obtained by culturing embryoid body cells with addition of for example BMP4, bFGF and CHIR, optionally as indicated in FIG. 1A. A method that can be used is described in Example 4. Other methods that produce KDR+CD56+CD34− mesoderm may be used, for example mesoderm produced by BMP/ACTIVIN specialized concentrations designed akin to cardiogenic mesoderms, also without CHIR addition and with for example a day 2 TGFbeta signaling blockade can also be used.

The embryoid body cells can be obtained by culturing human pluripotent stem cells. The human pluripotent stem cells can be embryonic human stem cells or induced human pluripotent stem cells.

Methods for making induced pluripotent stem cells (i.e. pluripotent stem cells artificially derived (e.g., induced or by complete reversal) from a non-pluripotent cell, typically an adult somatic cell) are known. For example, iPSC by inducing expression of one or more genes (including POU4F1/OCT4 (Gene ID; 5460) in combination with, but not restricted to, SOX2 (Gene ID; 6657), KLF4 (Gene ID; 9314), cMYC (Gene ID; 4609), NANOG (Gene ID; 79923), LIN28/LIN28A (Gene ID; 79727)).

In some embodiments, the starting population, e.g pluripotent stem cells, mesoderm etc, comprise cells expressing a marker such as a fluorescent marker. In other embodiments, the starting population e.g pluripotent stem cells, mesoderm etc, comprise cells expressing a geneticly encoded suicide system as a safety mechanism.

In one embodiment the method further comprises isolating a CD34+ population. Also provided is a method for producing a population of venous angioblast cells wherein the method further comprises isolating a CD34+ population from the cell population comprising CD34+ CD73+CD184− venous angioblast cells, optionally using a CD34 affinity reagent, CD31 affinity reagent and/or CD144 affinity reagent.

The CD34, CD31 and/or CD144 affinity reagent may be used alone or with or without a CD73 affinity reagent.

As shown in Example 1, isolating a CD34+ population of cells after specifying for venous angioblasts using a venous angioblast specifying concentration of VEGF, isolates a population of cells that increased for CD184-CD73+ cells, readily engraft and robustly produce sinusoidal endothelial cells when introduced into an animal. These cells can also be in vitro differentiated to a SEC like cell. As shown in FIG. 1B, isolated CD34+ cells show an increased level of KDR, SOX17, PECAM1 (CD31) and NFATC, and as shown in FIG. 1C, 2E, the CD34+ population corresponds to about 10.4+/−0.9% of the total day 8 population. Sorting this population for CD34 positive cells provides a highly enriched venous population of CD73+CD184− endothelial cells. Other markers can be used to obtain this population including for example CD31 and/or CD144.

The CD34 affinity reagent can be CD34 antibody conjugated beads, the CD31 affinity reagent can be CD31 antibody conjugated beads, the CD144 affinity reagent can be CD144 antibody conjugated beads and the CD73 affinity reagent can be CD73 antibody conjugated beads.

In one embodiment, the beads are magnetic beads for magnetic based separation of cells, optionally polystyrene spherical beads that are superparamagnetic. In some embodiments, one or more enrichment steps are performed.

Other methods of enriching or isolating can also be used. For example, cells can be isolated by Fluorescence Activated Cell Sorting (FACS) purification.

In one embodiment, the isolated CD34+ population comprises at least or about 50%, at least or about 60%, at least or about 70%, at least or about 80%, at least or about 85% or at least or about 90% CD34+ CD73+CD184− venous angioblast cells.

In some embodiments, the method is directed to producing venous endothelial cells from the venous angioblasts. As shown in FIG. 2A, culturing the CD34+ venous angioblasts for 1 or more days in an endothelial specifying concentration produces a population of cells that express one or more SEC markers.

Accordingly another aspect provided is a method of producing a population of venous endothelial cells comprising culturing KDR+CD56+CD34− mesoderm in a venous angioblast inducing media comprising for example a Notch inhibitor or a MEK inhibitor, a venous angioblast specifying concentration of VEGF and/or bFGF, until a cell population comprising CD34+ CD73+CD184− venous angioblast cells are obtained; isolating a CD34+ population from the cell population comprising CD34+ CD73+CD184− venous angioblast cells, optionally using a CD34 affinity reagent, CD31 affinity reagent and/or CD144 affinity reagent, optionally with or without a CD73 affinity reagent; and culturing the isolated CD34+ population in a venous endothelial inducing media comprising a venous endothelial specifying concentration of VEGFA to produce venous endothelial cells, optionally wherein the venous endothelial cells comprise PDGFRβ negative CD73 positive venous endothelial cells.

Referring to FIG. 2D, it is seen that the venous population produced using this method comprises a mixed population of CD34+CD31+ and CD34−CD31− cells. The venous populations expresses CD73. The CD34+ cells (which are generally CD31+) comprise venous endothelial lineage cells whereas the CD34− cells (which are also generally CD31− and PDGFRβ expressing) comprise for example mesenchymal and/or fibroblast cells.

In one embodiment, the isolated CD34+ population is cultured in a monolayer. Culturing in a monolayer can promote transition from angioblast to endothelial cell.

The venous endothelial cells comprising CD73+ venous endothelial cells have for example decreased but sustained expression of NRP2 and/or APLNR and elevated expression of CD34 and CD31/PECAM1 compared to the CD34+ CD73+CD184− venous angioblast cells without upregulation arterial markers such as NRP1, EFNB2.

As demonstrated in FIG. 2J, the venous population when cultured in venous endothelial inducing media for 4 days, show increased expression of venous fate markers such as NRP2, ALPNR, CD73, EPHB4, NR2F2, CD34 as well as STAB2 and decreased expression of arterial markers compared to arterial specified cells. Any of the markers listed in FIG. 2J can be used for example to confirm that differentiation is proceeding as expected, for example by removing a quantity of the cells being cultured and assessing for expression levels of one or more of the genes listed therein (transcript or protein levels).

In one embodiment, the venous endothelial specifying concentration of VEGFA is a concentration of from about 0 to about 30 ng/mL, optionally from about 0-20 ng/mL or any 0.1 increment from 0.1 to 29.9 ng/mL. In another embodiment, the venous endothelial specifying concentration of VEGFA is 0 ng/mL meaning that no exogenous VEGFA is provided instead relying on endogenously produced VEGFs.

The venous endothelial specifying concentration of VEGFA used in a method can be the same or different then the venous angioblast specifying concentration of VEGFA.

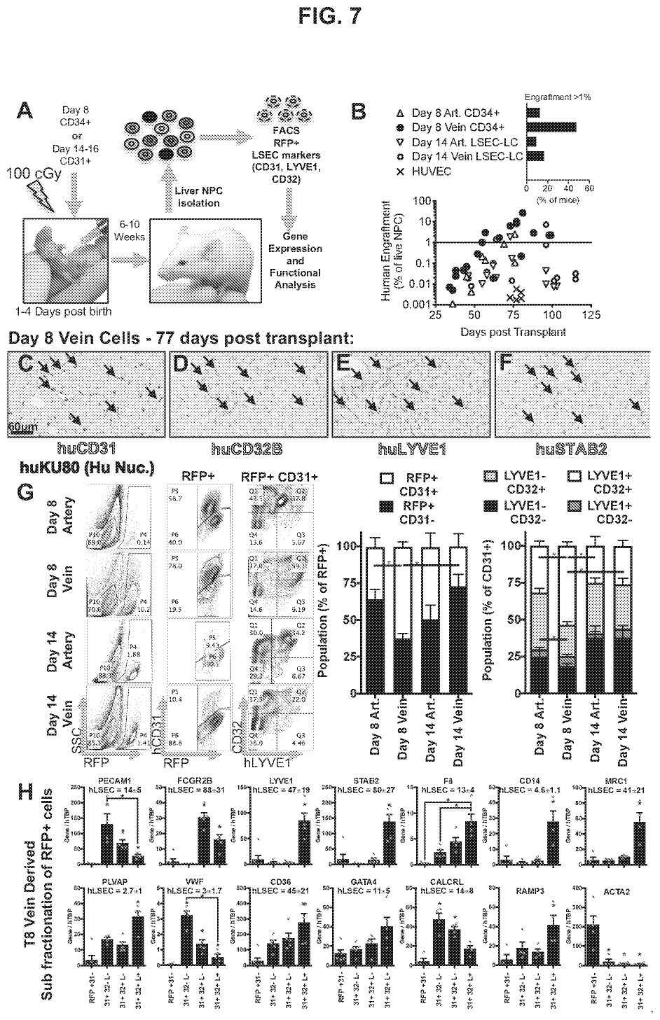

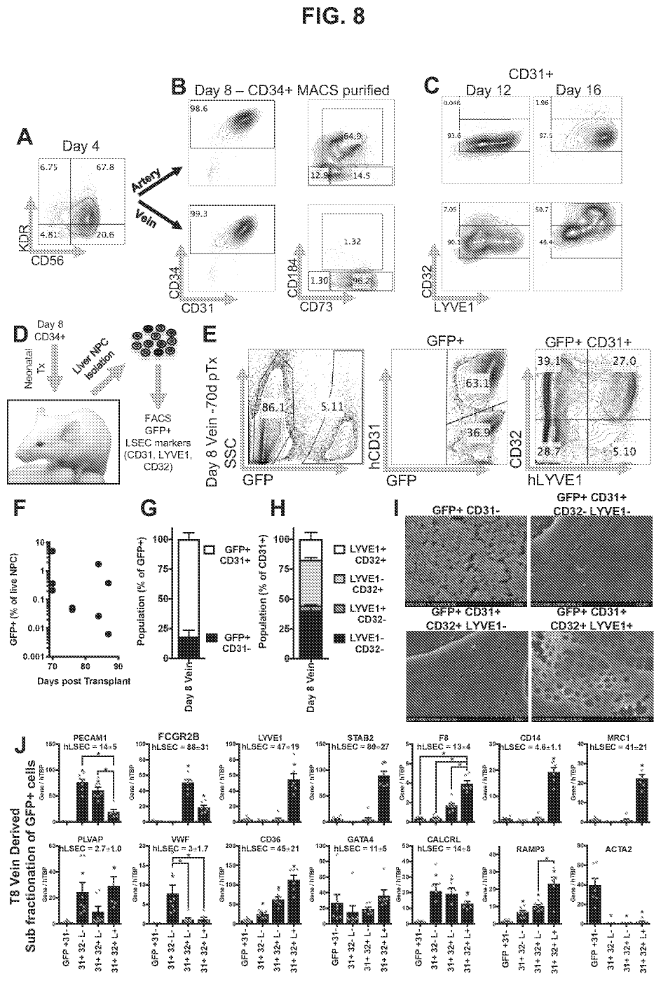

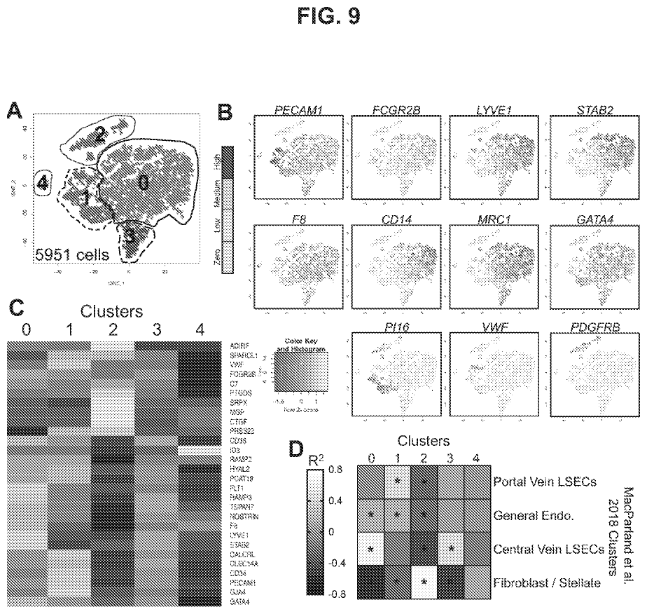

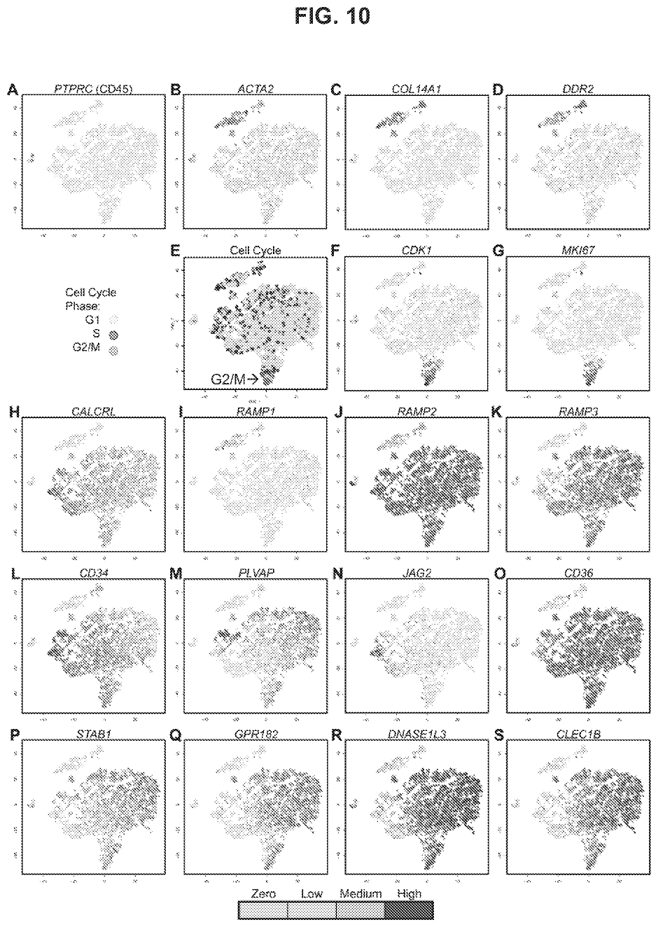

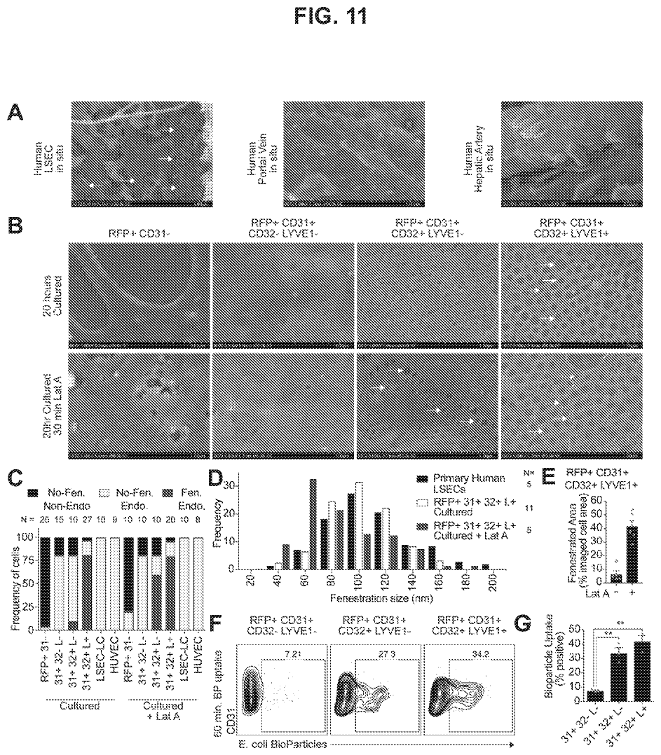

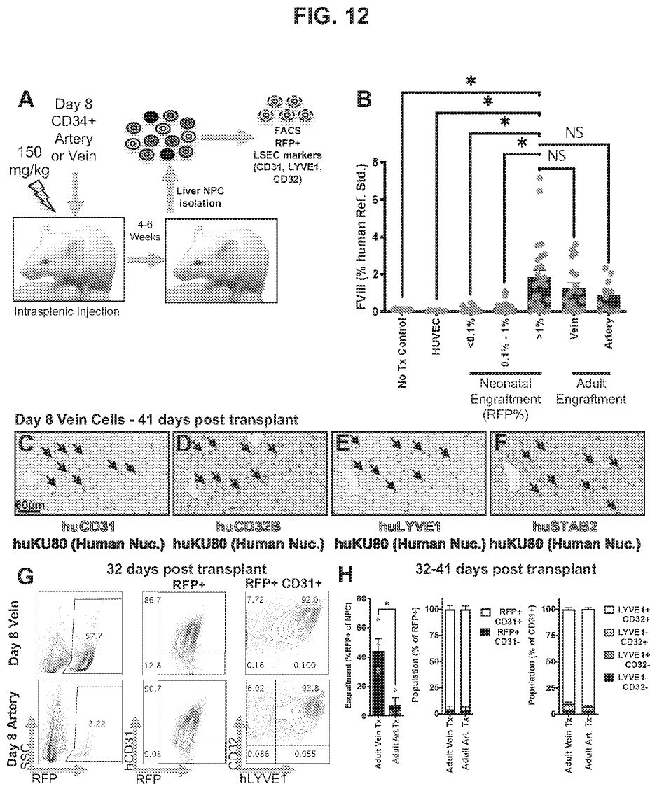

In one embodiment, culturing in the venous endothelial inducing media is for at least about 1 day, optionally about 1 to about 18 days (e.g., 1 to about 8 days, or any number of days between 1 and 18 days). For example, Example 1 and FIG. 7 show that cells cultured in venous endothelial inducing media for 6 to 8 days (e.g. day 14 to 16 of the differentiation method starting from PSC) were able to engraft and were detectable 40-100 days post transplant.

In addition, these cells start to show increased levels of SEC markers as well. As shown in Example 1, these endothelial cells can be further cultured in a SEC inducing media to produce SEC like cells.

Accordingly, a further aspect includes a method of producing sinusoidal endothelial cell like cells (SEC-LCs) comprising obtaining a population comprising CD34+CD73+CD184− venous angioblast cells; and differentiating the CD34+ CD73+ CD184− venous angioblast cells in vitro to obtain SEC-LCs.

In one embodiment, the CD34+ CD73+ CD184− venous angioblast cells comprise at least or about 50%, at least or about 60%, at least or about 70%, at least or about 80% or at least or about 90% of the population. As demonstrated in Example 1, the inventors were able at day 8 isolate a population of cells that was about 88% CD73+ and CD184− (FIG. 2G).

Methods and components described above can be utilized to produce the SEC-LCs. For example as described above, the population comprising CD34+ CD73+ CD184− venous angioblast cells can be obtained for example by culturing KDR+CD56+CD34− mesoderm in a venous angioblast inducing media having a venous angioblast specifying concentration of VEGF, until CD34+ CD73+ CD184− venous angioblast cells are obtained and isolating a CD34+ population, optionally using a CD34 affinity reagent CD31 affinity reagent and/or CD144 affinity reagent, optionally with or without a CD73 affinity reagent. Accordingly, in some embodiments, the population comprising CD34+CD73+CD184− venous angioblast cells is obtained according to a method described herein.

In one embodiment, the differentiating comprises: culturing the population of cells comprising CD34+ CD73+ CD184− venous angioblast cells in a venous endothelial inducing media comprising a venous endothelial specifying concentration of VEGFA, optionally wherein the population of cells is cultured in a monolayer system, to generate venous endothelial cells comprising PDGFRβ negative CD73 positive venous endothelial cells having decreased expression of NRP2 and/or ALPR compared to the CD34+ CD73+ CD184− venous angioblast cells; and culturing the venous endothelial cells comprising PDGFRβ negative CD73 positive venous endothelial cells in SEC inducing media to obtain SEC-LCs.

Culturing the venous epithelial cells comprising PDGFRβ negative CD73 positive venous endothelial cells in SEC inducing media can be for at least 1 day, optionally about 1 to about 18 days, preferably 1 to about 8 days or any number of days from 1 to 18 days.

Referring to FIG. 3, it is shown that venous endothelial cells cultured in SEC inducing media for about 4 days express one or more SEC markers.

Accordingly in some embodiments, the venous endothelial cells are cultured in SEC inducing media until one or more SEC markers—surface receptors, secreted proteins, or transcription factors associated with SECs is induced. Such SEC markers include, without limitation, LYVE1, CD14, CD32B (FCGR2B), CD36, CD54 (ICAM1), CD73, CD144, CD206 (MRC1), STAB1, STAB2, CLEC1B, PLAVP, GATA4, and F8, among others, and/or vascular markers such as CD31(PECAM1), CD34, CD105, CD309 (KDR), and VWF are reduced.

In one embodiment, the venous endothelial cells are cultured in SEC inducing media until one or more of LYVE1, STAB2, CD32B/FCGR2B, and F8 is expressed.

FIG. 3B demonstrates that SEC inducing media comprising a TGFbeta signaling inhibitor induces expression of one or more SEC makers.

In one embodiment, the SEC inducing media comprises a TGFbeta signaling inhibitor. In one embodiment, the TGFbeta signaling inhibitor is SB431542 (Tocris Bristol UK). Other TGFbeta signaling inhibitors sharing similar specificity (e.g. similar IC₅₀ and TGF signaling binding profiles) can be used (e.g., kinase inhibitors such as SB-505124, SB525334, LY2109761, LY2157299, LY580276, LY364947, or GW788388).

The SEC inducing media may comprise other SEC specifying components. The various components described herein can be added to the media daily and/or the media comprising the component can be replenished daily. In other embodiments, the component(s) or media comprising the component(s) is added to the cells every 2 days or only once during the particular culture period, for example up to 6 days.

For example, the addition of components can be by direct addition to the cells in media or by replacing the media with new media containing components. Components can for example be replaced daily from day 1-3 and replaced every 2 days from day 4 onwards.

The method may involve for example removing all media and replacing with media comprising the particular components depending on the stage of differention. For example, culturing in SEC inducing media can comprising removing all media every two days and replacing with SEC inducing media comprising components such as 30 ng/ml bFGF 6 μM SB-431542 and 1 mM cAMP.

It is also demonstrated herein that cAMP agonists can increase the in vitro specification of SEC cells. Accordingly, in some embodiments, the SEC inducing media comprises a cAMP agonist.

In one embodiment, the SEC inducing media comprises a TGFbeta signaling inhibitor and a cAMP agonist, and is optionally deficient in VEGFC.

The SEC-LCs can be isolated for example using a CD31 affinity agent. Accordingly, in some embodiments, the method further comprises isolating CD31+ SEC-LCs.

It is expected that cAMP and cAMP agonists would increase specification to SEC cells in other methods, for example where the starting population is an arterial endothelial population or a mixed population.

Accordingly a further aspect includes a method of producing SEC-LCs comprising culturing angioblasts to obtain arterial and/or venous endothelial cells; and differentiating the arterial and/or venous endothelial cells in the presence of a cAMP agonist to produce the SEC-LCs.

The angioblasts can for example be arterial angioblasts, venous angioblast or a mixed population of arterial and venous angioblasts.

In one embodiment, the arterial and/or venous angioblasts are an isolated population of venous angioblast cells obtained according to the method described herein.

In some embodiments, the cAMP agonist is cAMP or a cAMP analog such as 8-Br-cAMP, dibutyryl cAMP or Sp-8-Br-cAMPS. In other embodiments the cAMP agonist is a cAMP activator such as forskolin, IBMX and/or rolipram, optionally forskolin and IBMX. In some embodiments, the cAMP agonist is a cAMP analog and a cAPM activator, or multiple cAMP analogs and/or activators.

As further described in the Examples and shown in FIG. 3A, venous SEC-LCs are induced in media comprising TGFbeta signaling inhibitor, SB-431542 and cAMP analog on day 12. SEC-LCs begin to appear on day 13, and were confirmed to be present on days 14-22, with peak purity on days 14-16 from initiation with PSCs. Arterial SEC-LCs were observed at various purities between the same range of days e.g. 14-22 but their peak purity was found to be around days 16-18, compared to days 14-16 for venous SEC-LCs.

Accordingly, culturing the venous endothelial cells comprising PDGFRβ negative CD73 positive venous endothelial cells in SEC inducing media to obtain SEC-LCs and/or differentiating the arterial and/or venous endothelial cells in the presence of a cAMP agonist to produce the SEC-LC can comprise for example at least 1 day to up to 18 or more days, optionally at least 1 day, at least 2 days, at least 3 days or any number of days and/or parts thereof between and including 1 day and 18 days.

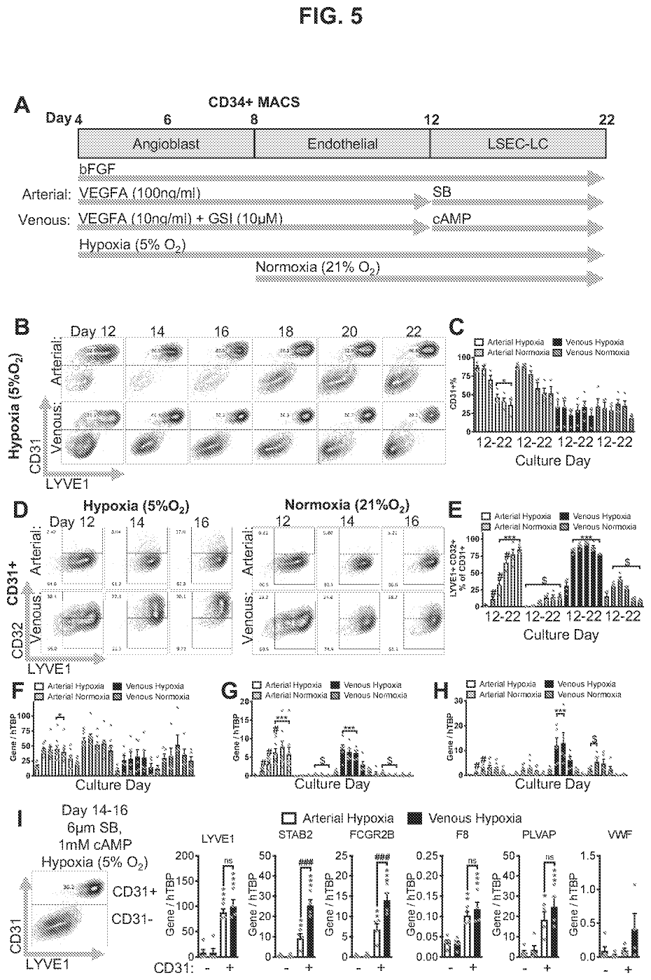

It is also demonstrated herein that hypoxic conditions can increase the expression of CD32B/FCGR2B in both artery and venous CD31⁺LYVE1⁺ populations; however, the upregulation of expression occurred 2-4 days earlier in the venous than in the arterial cells (FIG. 5D and 5E).

Accordingly, in some embodiments, one or more of the culturing steps is performed under hypoxic conditions. For example one or more of the CD34+ CD73+ CD184− venous angioblast cells, the venous endothelial cells, the SEC-LCs or the isolated CD31+ SEC-LCs are cultured under hypoxic condition, optionally wherein the hypoxic condition is a cell culture incubator environment of 5%CO₂/5%O₂ and/or addition of a hypoxia inducible factor (HIF) prolyl-hydroxylase (PHD) inhibitor (HIF-PHDI).

Various HIF-PHDIs are known. In one embodiment, the HIF-PHDI is a tricyclic triazole compound, optionally IOX2, IOX4, DMOG or similar compounds that also increase HIF 1 a signaling. In another embodiment, the HIF-PHDI is selected from Daprodustat (2-[(1,3-dicyclohexyl-2,4,6-trioxo-1,3-diazinane-5-carbonyl)amino]acetic acid), Molidustat (2-(6-Morpholin-4-ylpyrimidin-4-yl)-4-(triazol-1-yl)-1H-pyrazol-3-one), Roxadustat (2-[(4-Hydroxy-1-methyl-7-phenoxyisoquinoline-3-carbonyl)amino]acetic acid), Vadadustat (2-([5-(3-Chlorophenyl)-3-hydroxypyridine-2-carbonyl]amino)acetic acid) and Desidustat (2-[1-(Cyclopropylmethoxy)-4-hydroxy-2-oxoquinoline-3-carbonyl]aminolacetic acid).

Another aspect relates to a method of producing arterial angioblasts, arterial endothelial cells etc. In such methods, a CD34 population is isolated after angioblast specification without for example inhibition of NOTCH signaling and an arterial specifying and proliferating concentration of VEGFA is used instead of a venous specifying concentration.

In some embodiments, the methods comprise one or multiple purification rounds for example one or more multiple FACS purifications based for example on cell surface expression patterns described herein.

In one embodiment, the population is an isolated population and/or an in vitro produced population. For example, an aspect includes an in vitro produced cell according to a method described herein, such as an in vitro produced SEC-LC, an in vitro produced venous angioblast.

Yet a further aspect includes a population of cells comprising an in vitro produced SEC-LC and/or in vitro produced venous angioblast and/or other cell described herein, optionally isolated.

As demonstrated herein, SEC-LC cells produced according to the methods described share similarities with in vivo SECs. Notably, the in vitro derived SECs have decreased F8 levels as well as other differences compared to in vivo SECs.

In an embodiment, the population comprises at least 50%, at least 60%, at least 70%, at least 80% or at least 90% of cells expressing one or more markers described herein.

A further aspect includes a composition comprising the in vitro produced cells described herein for example comprising venous angioblasts, venous endothelial cells and/or SEC-LCs described herein.

The composition can comprise for example a diluent or a carrier. Suitable diluent includes for example a suitable culture medium, or freezing medium containing for example serum, a serum substitute or serum supplement and/or a suitable cryoprotectant such as dimethyl sulphoxide (DMSO), glycerol Methylcellulose or polyvinyl pyrrolidone. In some embodiments the diluents are sterile.

In one embodiment, the carrier is a pharmaceutically acceptable carrier. The in vitro produced cell or population of cells may be comprised in a vial such as a sterile vial.

Another aspect includes a kit comprising one or more of a cell or population of cells produced herein or a vial comprising said cell or population of cells, one or more inducing or specifying components for producing cells described herein and/or instructions for producing one or more cells described herein.

The kit may include for example one or more of cells produced herein, optionally in freezing media and packaged in a coolant such as dry ice or liquid nitrogen, matrigel or equivalent ECM coated screening plate, basal growth media, one or more components described herein (e.g. bFGF, VEGF), SEC induction components (e.g., bFGF, cAMP, SB, IOX2), isolation antibodies and/or quantification antibodies, for example for use in FACs.

### Uses

In vitro and in vivo uses are also provided in other aspects.

In vitro produced cells described herein including venous angioblasts, venous endothelial progenitors and SEC-LCs can be used in a screening assay to pre-screen candidate drugs.

Accordingly, one aspect is a screening assay comprising contacting a cell population produced according to a method described herein with a test compound; and measuring a desired read out, such as the ability and/or rate of the cells to scavenge a test compound and/or toxicity of the test compound on the cells, compared to the cells treated with a vehicle control.

LSEC-LCs (or partially mature LSEC-LCs, defined as expressing FCGR2B and other LSEC markers but lacking, e.g., mature F8 gene expression levels) can be used, for example, in vitro for testing compounds (e.g., drug candidates) for scavengability by human LSEC-like cells and for toxicity to human LSEC-like cells. Such screening methods can be used on pure or pooled human PSC-derived backgrounds, allowing population- or ethnicity-specific pharmacology testing and prevention of adverse drug reaction; and/or for testing and optimization of biological drug candidates (e.g., monoclonal antibodies (e.g., therapeutic antibodies), cytokines, small molecules, etc.) for LSEC-associated binding and scavenging characteristics (e.g., to achieve improved and/or desired biodistribution and circulation half-life). Such assays can be performed with the goal of identifing test compounds that exhibit minimal or reduced binding to, or scavenging by, LSEC or, alternatively, test compounds that exhibit increased binding to, or scavenging by, LSEC. For therapeutic drugs that are intended to act outside the liver (e.g., a therapeutic compound for treating breast cancer), it is generally desired to minimize the scavenging of such a compound by the liver SEC-LCs. In cases in which it is desirable to concentrate a compound in the liver (e.g., hepatocyte proliferation drugs, hepatic fibrosis blocking drugs, hepatocellular carcinoma targeting oncotheraputics, etc.), then increased scavenging mechanisms by SEC-LCs are desirable. Accordingly, such screening methods may play a major role in identifying compounds that are appropriately scavenged, or not, in vivo by the liver. See, for example, Sorensen et al. (2015, Compr. Physiol., 5(4):1751-74) for a table of biomolecules that can be scavenged by LSECs. Significantly, the diversity of molecules that LSECs bind makes them an extremely valuable screening platform, so as to either avoid unwanted and unexpected scavenging mechanisms or exploit the scavenging mechanism and target a therapeutic to the liver.

Animal models comprising in vivo matured SEC-LCs can be used as humanized models for in vivo drug testing. Accordingly another aspect provides a humanized animal model comprising engrafted in vitro produced cells described herein, optionally venous angioblasts produced according to a method described herein, venous endothelial cells produced according to to a method described herein and/or SEC-LCs. In some embodiments, the in vitro produced cells are produced from human starting cells (e.g. CD34+CD73+CD184− venous angioblast cells, KDR+CD56+CD34− mesoderm, embryoid body cells or pluripotent stem cells) comprising a marker, such as a fluorescent marker, light emitting marker etc for tracking the human in vitro produced cells in the animal model. The animal is for example a rodent such as a mouse or a rat. In one embodiment, the animal model is a mouse with humanized liver vasculature wherein cells produced according to a method described herein are injected into the mouse's liver and allowed to engraft. Said humanized vasculature mice can be used to test drug toxicity and ADME/TOX. As they would comprise human blood vessels, they may provide a more suitable model.

In another embodiment, the animal model is produced where the model is lacking a key blood coagulation factor (e.g., F8 or VWF are existing mouse models). Disease model mice are rendered immunocompromised by breeding to standard mouse strains (NSG, NOD-Scid, Nu/Nu, RAG1−/− etc) or by administration of immunomodulatory therapeutic cocktails. Immunocompromized disease model mice can be transplanted by intrahepatic injection in neonates, via splenic injection in adults or in ectopic transplantation sites. Engrafted disease model mice are expected to have corrected disease phenotypes (normalized blood clotting times, circulating clotting factor levels) with sufficient engraftment providing evidence for cell based therapies as well as testing methods to of other pharmacological therapies to enhance subtherapeutic F8/VWF levels to therapeutic ranges.

In one embodiment, the animal model is an immunocompromised non-disease mouse where fibroblast mediated cirrhotic liver disease states can be modeled by hPSC-derived non-endothelial outgrowths. In such a model, mice are injected with venous angioblasts and/or other populations described herein (and such cells engraft) to cause fibrosis and the model used to screen for or test potential liver disease treatments. In this way, novel therapeutic strategies can be examined for their ability to slow, reverse, or cure human fibrotic liver disease.

Also provided in another aspect is a cellular therapy. In vitro produced cells can be produced and prepared as pharmaceutical compositions. In one embodiment, the method comprises introducing a population of cells (e.g., venous angioblast, venous endothelial, CD34+ isolated, SEC-LCs, CD31+ isolated etc) produced in vitro according to a method described herein, or a composition comprising said cells, into a subject in need thereof. In one embodiment, the population of cells or composition is introduced into the subject by injection. In another embodiment, the population of cells or composition is comprised in a device, such as an immunoisolation device, vascular engraftment device, or multi cellular transplantation device, such as Encaptra® cell delivery system by ViaCyte.

In one embodiment, the population or composition is introduced into the subject in the liver, bone marrow, spleen or anterior pituitary representing other SEC containing tissues.

As demonstrated in the Examples, in vitro produced angioblast, endothelial cells and SEC-LCs after engrafting produce increased levels of F8, the coagulation factor deficient in Hemophilia A. In one embodiment, the cellular therapy is for treating a liver disease such as Hemophilia A. This therapy could be delivered as a direct injection of cells to the liver, other SEC containing tissues in the body, or at an ectopic transplantation device with or without a biocontainment immunoisolation device. Since even modest production of F8 has improved patient outcomes, the therapeutic range from the engrafted cells is broad. Expected outcomes include decreased spontaneous bleeding episodes, shortened bleeding times during traumatic injuries and decreased total mortality.

In another embodiment, the cellular therapy is for VWF-disease (similarly to F8-deficiency), acute and chronic liver damage optionally from hepatotoxicity due to drug induced liver injury, alcoholic and non-alcoholic steatohepatitis (fatty liver disease or NASH) or other progressive cirrhosis diseases or disorders.

In some instances, the SEC-LCs described herein also can be used to treat a liver disease such as hepatocellular carcinoma (HCC). The LSECs around a liver cancer foci are altered in terms of expression patterns (decreased LYVE1, STAB1, STAB2, CD32B), resulting in reduced scavenger activity, and loss of fenestration as the LSECs are cappillarized. Therefore, these altered LSECs do not deliver therapeutics to the region of the cancer in the same way as “normal” LSECs. It's possible that if the altered LSECs are replaced or supplemented with functional LSECs (e.g., LSECs that deliver chemotherapeutics more efficiently), treatments may be more effective.

Cell therapy also can be a supportive cell therapy. For example, the cells described herein can be administered with hepatic cells (e.g. heptatocytes and/or cholangiocytes) and/or hematopoietic cells and introduced into the liver. Similarly, these cells can be administered with hematopoietic, endocrine, and or stromal cell types or derivatives and administered to bone marrow, spleen or the anterior pituitary respectively.

The SEC-LCs described herein produce Factor VIII (expressed from the F8 gene). In some instances, it may be desirable to monitor the SEC-LCs for expression of F8 or for the presence of Factor VIII. Such monitoring can take place in culture (e.g., in vitro) or in vivo following administration of such cells to a subject. For example, an ELISA from Affinity Biologicals Inc. (Catolouge number FVIII-AG) can be used to detect FVIII protein.

### EXAMPLES

### Example 1

Specification of Artery and Venous Angioblasts from PSCs

For these studies, hPSCs were differentiated using previously described embryoid body (EB)-based protocol optimized for the generation of hematopoietic derivatives (Sturgeon et al., 2014). With this protocol, mesoderm is induced through the addition of BMP4 (days 0-4), bFGF (days 1-8) and CHIR (days 2-4) to the EB cultures as indicated in FIG. 1A. At day 4 of differentiation, BMP4 and CHIR are removed from the cultures and replaced with VEGF. Under these conditions, as POU5F1 (OCT4) expression wains, a primitive streak population, defined by the upregulation of T (BRACHYURY) and MESP1 (FIG. 1B) is detected by day 2 of differentiation and KDR+CD56+CD34− mesoderm is induced by day 4 (FIG. 1C). This mesoderm did not highly express CD235a/b indicating that it represented the equivalent of the population that will give rise to definitive hematopoiesis. Concomitant with the peak expression of the endothelial progenitor marker ETV2 at day 4, endogenous VEGFA upregulation marked the initiation of KDR+CD34+ angioblasts at day 5 (FIG. 1B). By day 8 of differentiation, a CD34⁺CD31^{low} population emerged that expressed the vascular genes, KDR, SOX17 AND NFATC (FIG. 1B).

Previous work from our group (Ditadi et al., 2015) and others (Zhang et al., 2017), has shown that VEGF, bFGF, NOTCH signaling play a role in the specification of arterial and venous vascular fates in hPSC differentiation cultures. The effects of VEGF signaling in promoting arterial and venous development were assessed. To address this, different concentrations of VEGF (0 to 100 ng/ml) were added to day 4 cultures that contained low amounts of bFGF (5 ng/ml) and the resulting day 6 populations were analyzed for the presence of CD34⁺CD31⁺ vascular cells and CD184⁺CD73^{low}CD34⁺ and CD184⁻CD73⁺CD34⁺ populations that we have previously shown contain progenitors specified to arterial and venous fates respectively. Increasing VEGF concentrations led to a significant and dose dependant increase in the frequency of CD34+ cells (FIG. 1D). Additionally, higher amounts of VEGF biased the cells away from a CD184⁻CD73⁺ venous-like population detected in cultures that received no VEGF or 10 ng/mL VEGF to a predominantly CD184⁺CD73^{low} arterial-like population in cultures treated with 100 ng/ml. Manipulation of bFGF concentrations (up to 30 ng/ml) in cultures containing high amounts of VEGF (100 ng/ml) led to a modest increase in the size of the total CD34⁺ population (between days 4-6 of differentiation), a decrease in the size of the CD34⁺CD184⁺CD73^{low} subpopulation and an increase in the size of the CD34⁺CD184⁻CD73⁻ subpopulation (FIG. 1D). Although exogenous bFGF did not have a dramatic effect on the development of the different endothelial populations, signaling through this pathway (30 ng/ml) is required and benificial to sustain CD34 frequency for further enrichment.

To further characterize the putative arterial and venous populations, the expression patterns of genes that are known to be differentially expressed in these vascular cell types were analysed. For these studies, arterial progenitors identified as CD34⁺CD184⁺CD73^{low} cells were generated by specification of mesoderm with high concentrations of VEGF (100 ng/ml) whereas CD34⁺CD184⁻CD73⁺ venous progenitors were specified with lower concentration of VEGF (10 ng/ml) and inhibition of the NOTCH pathway through the addition of GSI between days 4 and 8 of differentiation as previously described (Ditadi et al., 2015; Zhang et al., 2017) (FIG. 2A-C, E-G). At day 8, both CD34+ angioblast populations were isolated by MACS and cultured as monolayers for an additional 4 days allowing proliferative angiogenesis to generate CD34⁺CD31⁺ endothelial cells (FIG. 2D). During this culture period, the majority of the arterial population retained CD34 and CD31 expression and upregulated CD184 expression (FIG. 2H). By contrast, approximately 40% of the venous population lost CD34 and CD31 expression and upregulated expression of the mesenchymal marker PDGFRbeta during this time frame. These PDGFRbeta positive cells are mesenchyme/fibroblasts. The CD34⁺CD31⁺ population that persisted retained high levels of CD73 expression and did upregulate CD184 (FIG. 2I), a pattern similar to vein-derived, VEGFA-induced, NOTCH-mediated angiogenesis found in migrating venous tip vascular cells in vivo (Hasan et al., 2017). qRT-PCR expression analyses (FIG. 2J-2L) revealed that the day 8 arterial angioblasts expressed higher levels of the arterial makers EFNB2, CXCR4, NRP1, DLL1 and the components of the NOTCH signaling pathway HEY1, HEY2, and HES5 than the corresponding day 8 venous population. The venous angioblasts expressed higher levels of markers associated with a venous fate (EPHB4, CD73, NRP2, NR2F2, APLNR, STAB2) than the arterial population. These cells also expressed higher levels of KDR and FLT4 but not lymphatic markers (PROX1, PDPN) or early expression of LSEC markers (LYVE1, CD32B, F8). The arterial population maintained elevated levels of EFNB2, CXCR4, NRP1, HESS and HEY1 expression following the 4-day expansion culture whereas the cultured venous population expressed the venous markers EPHB4, CD73, NRP2, NR2F2, APLNR, STAB2 during this time. Taken together, these findings confirm that it is possible to generate arterial and venous vascular cells through the stage specific modulation of the VEGF and NOTCH signaling pathways.

In Vitro Specification of LSEC-LCs from Arterial and Venous Endothelial Lineages

The specification of endothelial cells to LSEC-like cells (LSEC-LCs) has been suggested to involve a number of signaling pathways including TGFbeta (Arai et al., 2011; Koui et al., 2017), Adrenomedullin/cAMP (Arai et al., 2011), and in vivo including VEGFC/VEGFR3 (Ding et al., 2010). To examine the role of these pathways in hPSC-derived LSEC specification, venous angioblast-derived populations were treated with different concentrations of 8-Br-cAMP (0, 0.1, 0.3, 1.0 mM), the TGFbeta signaling inhibitor SB431542 (0, 6, 18 μM), and VEGFC (0, 100 ng/ml) every 2 days for 4 days (FIG. 3A). The resulting bulk populations were analyzed for expression of LSEC associated markers by RT-qPCR (FIG. 3B). Based on immunofluorescence (Strauss et al., 2017), flow cytometry (Fomin et al., 2013), bulk RNA-seq (Shahani et al., 2014), and recent single cell RNA-seq of human liver (MacParland et al., 2018) studies, human LSECs are distinguished by the co-expression of surface receptors, secreted proteins, and transcription factors including, LYVE1, CD14, CD32B (FCGR2B), CD36, CD54 (ICAM1), CD73, CD144, CD206 (MRC1), STAB1, STAB2, CLEC1B, PLAVP, GATA4 and F8 among others. Additionally, they express lower levels of canonical vascular markers such as CD31(PECAM1), CD34, CD105, CD309 (KDR), and VWF than portal arterial and venous endothelial cells. As a candidate marker set we focused the analysis on expression of scavenger receptors LYVE1, STAB2, CD32B/FCGR2B, F8, and VWF. Addition of cAMP alone led to the upregulation of expression of LYVE1, STAB2 and FCGR2B while inhibition of TGFbeta alone induced the expression of STAB2 and FCGR2B but not LYVE1 (FIG. 3B). The combination of these two pathway modulators induced the highest expression levels of these genes as well as that of Factor VIII (F8). Although the addition of VEGFC was found to modestly enhance the expression of some LSEC markers, it also increased the expression of the lymphatic markers PROX1 and FLT4. Given this, we used the combination of SB (6 μM) and cAMP (1 mM) to induce the LSEC-like fate for the studies described below.

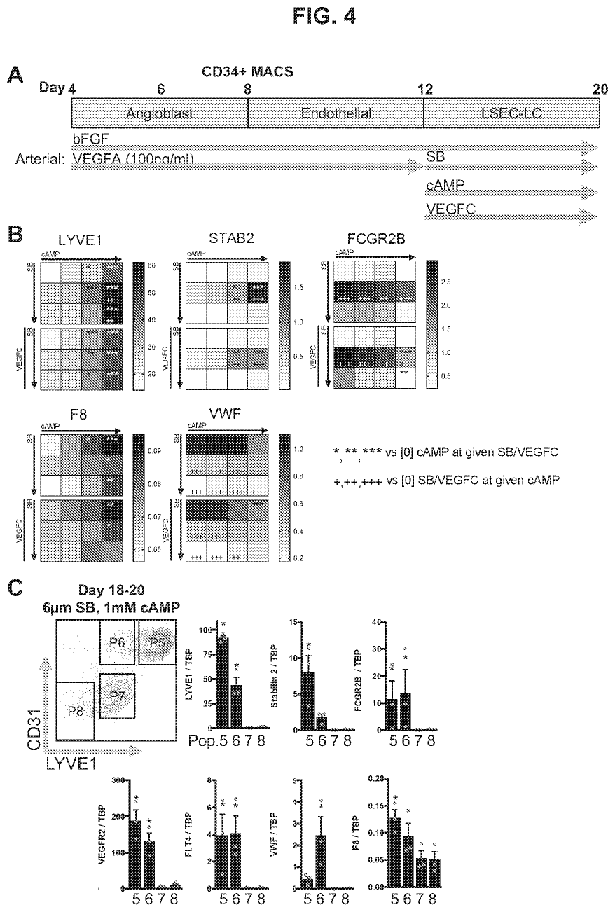

When induced with SB and cAMP for 4 days, the day 12 venous derived cells generated a population that contained both CD31⁺LYVE1⁺ and CD31⁻LYVE1⁻ subpopulations. Molecular analyses revealed that CD31⁺LYVE1⁺ cells expressed significantly higher levels of LYVE1, STAB2, FCGR2B and F8 than the CD31⁻LYVE1⁻ cells, indicating that this double positive subpopulation contains the hPSC-derived LSEC-like cells (FIG. 3C). While LYVE1, STAB2, and FCGR2B were induced to levels comparable isolated primary human LSECs (LYVE1 47±19; STAB2 80±27; FCGR2B 88±31; F8 13±4), F8 levels were over 160-fold lower. The combination of SB and cAMP also induced the expression of LSEC genes in arterial-derived endothelial populations (FIG. 3 and FIG. 4).

Mature blood-carrying hepatic vasculature includes both oxygen rich arteries and oxygen depleted veins and sinusoidal vessels. Previous studies have found that the portal vein and hepatic vein have oxygen contents of 7.22 and 6.70 kPa O₂ respectively which closely matches the 7.39 kPa O₂ recorded for 5%CO₂/5% O₂ equilibrated cell culture incubator environment. Similarly, the hepatic artery has been observed to contain 20.9 kPa O₂ which correlates to a 5% CO₂/95%air incubator media content of 18.46 kPa O₂ (Martinez et al., 2008). To determine if oxygen tension impacts endothelial specification toward the LSEC fate, we cultured arterial and venous angioblasts under hypoxic (5% O₂) or normoxic (21% O₂) conditions from day 8 of culture onward. SB and cAMP were added at day 12 of culture to induce the LSEC fate (FIG. 5A). Flow cytometric analyses revealed the presence of a CD31⁺LYVE1⁺ population in both the arterial and venous populations cultured under either hypoxic or normoxic conditions (FIG. 5B,C). Hypoxia did induce the expression of CD32B/FCGR2B in both artery and venous CD31⁺LYVE1⁺ populations; however, the upregulation of expression occurred 2-4 days earlier in the venous than in the arterial cells (FIG. 5D,E). qRT-PCR-based expression analyses of the total populations confirmed the flow cytometric analyses showing the hypoxia-induced upregulation of CD32B. Additionally, these analyses showed that hypoxia led to an upregulation of expression of STAB2 and that this effect was restricted to the venous populations (FIG. 5F-H). Molecular analyses of the hypoxia cultured, isolated CD31⁺LYVE1⁺ populations revealed that the venous cells expressed significantly higher levels of STAB2 and CD32B than the arterial cells (FIG. 5I).

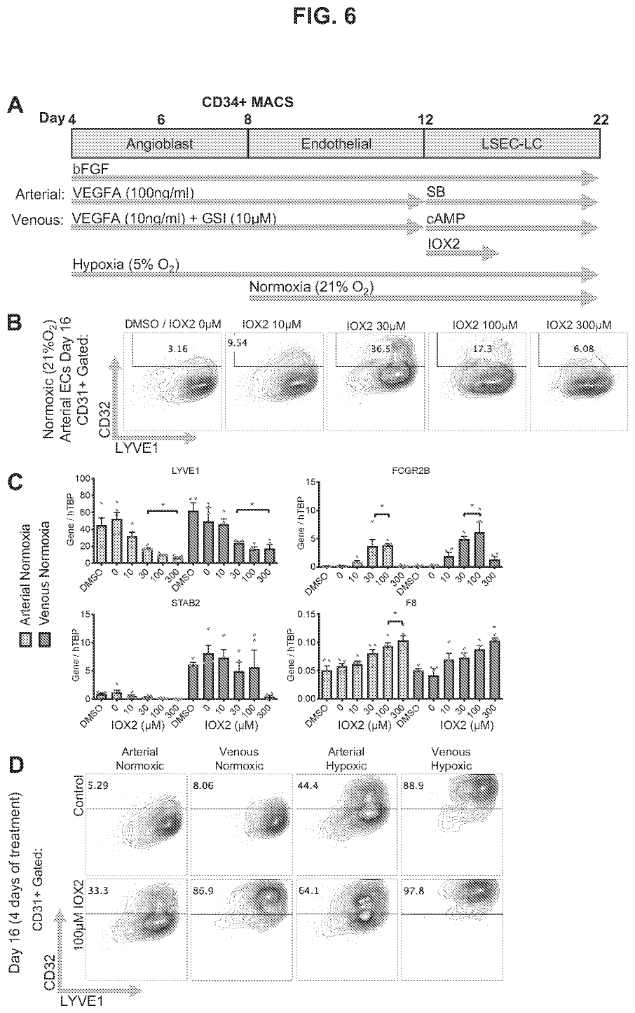

To further investigate the role of oxygen on LSEC marker expression, we next interrogated the HIFI a signaling pathway as a probable mechanistic regulator of EC gene expression. Application of IOX2, a specific PHD inhibitor that stabilizes HIF1a mediating elevated signalling, resulted in a dose dependant upregulation of CD32 protein in CD31⁺LYVE1⁺ arterial ECs cultured under elevated oxygen conditions (FIG. 6A,B). Agonism of HIF1a signaling significantly downregulated LYVE1, upregulated FCGR2B and F8 expression and did not change STAB2 expression in both arterial and venous ECs (FIG. 6C). Moreover, the 4-day application of IOX2 to cells cultured under hypoxic conditions resulted in a highly efficient conversion of CD31⁺ ECs to LYVE1⁺CD32⁺ LSEC-LCs by day 16. This hypoxic response was observed in both arterial and venous ECs, however the effect was more pronounced in venous cells indicating a hypersensitivity to HIF1a signalling (FIG. 6D).

Although our in vitro differentiation protocol does promote the development of a LSEC-like population with robust scavenger receptor expression, the levels of F8 are exceptionally low. To determine if an in vivo environment would support the development of bona fide hPSC-derived LSECs, we transplanted day 8 CD34⁺ angioblasts or day 14-16 CD31⁺ LSEC-LCs into the liver of 1-4 day old irradiated NSG neonatal mice (FIG. 7A). This model was chosen as the early postnatal growth period is one where the liver mass increases while the organ transitions from an immature fetal-like state to the mature liver. This developmental window supports both proliferation and differentiation of endogenous hepatic cells and potentially exogenous less mature hPSC-derived cell derivatives. To facilitate identification and recovery of human-derived cells, transplanted cells were generated from HES2 hESCs that were engineered to constitutively express tdRFP (Irion et al., 2007). Monitoring the frequency of tdRFP+ cells within the non-parenchymal cell (NPC) fraction showed that all hPSC-derived transplanted cell populations showed detectable levels of engraftment (FIG. 7B). Day 8 venous angioblasts showed significantly higher levels of engraftment compared to arterial angioblasts and both LSEC-LC populations. In contrast, HUVECs were not able to stably engraft NSG livers (<0.001% hCD31⁺, N=5 at day 73-80 days post transplant; FIG. 7B).

Histological examination of long term engrafted adult mice found a close correlation between the percentage of tdRFP+ engraftment and the observation of regions of NSG liver containing human cells (KU80⁺) (FIG. 7C-7F). These areas contained CD31+ cells that co-expressed CD32B, LYVE1, and STAB2. CD31− cells, possibly representing fibroblast or stellate like cells were also detected. These human derived endothelial/LSEC marker positive regions were consistently associated with central veins and surrounding sinusoidal vascular regions of the liver suggesting at least some fraction of the cells were hESC-derived LSECs.

To facilitate the characterization of the engrafted hPSC-derived cells we devised a simplified FACS based isolation strategy that was free from murine LSEC contamination. Namely, isolation of human cells by tdRFP⁺ recovery followed by endothelial fractionation with human-specific CD31 and sub-fractionation of endothelial cells with CD32 and human specific LYVE1 (FIG. 7A,G). This isolation method facilitated quantification of graft composition and recovery of hPSC-derived cell populations for comparison to similarly purified primary human LSECs (live cells, CD45⁻, CD68⁻, CD31⁺, CD32⁺). As all four transplanted hPSC-derived cell populations were capable of engrafting NSG livers, albeit at different rates, we monitored graft composition during cell isolation to determine if the transplanted cells showed engraftment bias. Day 8 venous angioblast-derived cells showed significantly elevated CD31⁺ endothelial cell populations which were predominantly CD32⁺LYVE1⁺ cells compared to other transplanted cell populations (FIG. 7G). Given this significant composition bias toward LSEC markers, we focused further analysis of sub-fractionated populations to day 8 venous angioblast-derived cells (FIG. 7H). hESC-derived endothelial that expressed CD31, CD32, and LYVE1 showed a unique upregulation of a large number of LSEC markers not seen in other subfractions (LYVE1, STAB2, F8, CD14, MRC1, RAMP3) and low expression of large vessel endothelial markers (PECAM1, VWF, CALCRL). In contrast, hESC-derived CD31⁺ cells that were negative for CD32 and LYVE1 (tdRFP⁺ CD31⁺ CD32⁻LYVE1⁻) expressed the highest levels PECAM1, VWF, CALCRL levels but lacked expression LSEC markers. hESC-derived CD31⁻ cells (tdRFP⁺ CD31⁻) did not express LSEC markers but instead showed elevated expression of ACTA2, PDGFRB, DDR2 and MGP suggesting a mixture of smooth muscle, fibroblast, or mesenchymal cell derivatives similar to in vitro derived CD31⁻ cells. These gene signatures of the CD31⁺ CD32⁺ LYVE1⁺ LSECs and other cell fractions was also replicated in venous angioblast derived LSECs from the H1-GFP hESC line (FIG. 8) that similarly showed comparable LSEC marker expression levels to primary human purified LSECs including adult levels of F8 gene expression which was notably absent in vitro (FIG. 5I, 7H). Furthermore, these data validate the isolation strategy, and suggests that hESC-derived LSECs generated though an in vivo-matured venous angioblast in vitro intermediate stage have gene expression levels comparable to isolated primary human LSECs (hLSEC).

To examine the graft composition in an unbiased manner and to facilitate direct comparison to primary human hepatic vascular cells (MacParland 2018 in press), we utilized scRNA-seq to profile the live, tdRFP+ cells, FACS purified from the NPC fraction isolated from a 77-day engrafted mouse initially transplanted with day 8 venous angioblasts. Of the 5,951 tdRFP+ cells profiled by scRNAseq, 5,258 (88.4%) were PECAM1+ endothelial cell as part of clusters 0, 1, and 3. Clusters 2 and 4 contained the remaining 693 (11.6%) PECAM1 negative cells (FIG. 9A-C). The most numerous PECAM1+ population, cluster 0, was defined by high expression of scavenger receptors and functional LSEC genes including F8. Cluster 0 with 3801 cells occupied 64% of the total tdRFP+ population and was characterized by elevated expression of LYVE1, STAB1, STAB2, FCGR2B, CD14, MRC1, CLEC1B, and DNASE1L3 but lower or absent expression of PECAM1, CD34, and VWF (FIG. 9B,C and FIG. 10). The second largest endothelial cluster (1005 cells, 17% of tdRFP+ cells), cluster 1, had little to no expression of scavenger expression genes seen in cluster 0 but instead had elevated PECAM1, CD34, VWF, PLVAP and AQP1. Cluster 3 was an endothelial cluster containing 452 PECAM1+ cells (7.6% of tdRFP+) with a mixture of expression patterns from cluster 0 and 1. Based on the cell cycle phase score high in G2M and elevated expression of MKI67, CDK1, CDKN3, PCNA, CCNB1, and ID1 (FIG. 10), cluster 3 is likely proliferating LSECs similar to those described in mice (Ding et al., 2010). Cluster 2 (592 cells; 10%) and 4 (101 cells, 1.7%) representing the majority of PECAM1 negative cells had a shared pattern of elevated expression of multiple collagens, ACTA2, PDGFRbeta, DDR2, POSTN, TGFB1, DES and others suggestive of fibroblasts, smooth muscle cells, and general mesenchymal derivatives (FIG. 10). We next compared these expression patterns to endothelial and fibroblast like clusters in primary human liver tissue (FIG. 9D). This comparison utilized the top 50 differentially expressed genes defined by MacParland et al. in the three endothelial and one fibroblast/mesenchyme cluster to concisely define the target populations (MacParland et al., 2018). This analysis showed a significant and strong positive correlation between cluster 0 and central vein associated zone ⅔ human LSECs (R²0.65, p=7e−19). No significant correlation was observed to portal region/zone 1 human LSECs (R²−0.03, p=0.7). Cluster 1 was significantly correlated to LSEC lice endothelial cells of the portal region/zone 1 of the liver (R²0.48, p=1e−9) and genera endothelial cells (R²0.18, p=3e−2) but not to central vein associated zone ⅔ human SLECs (R²−0.14, p=9e2−). Cluster 3 correlated to central vein LSEC populations (R²0.49, p=4e−10) which matched the predicted identify as a LSEC proliferative cluster. Cluster 2 showed a significant positive correlation to the fibroblast/stellated cell cluster (R²0.71, p=1e−23) and significant negative correlations to all three endothelial comparative clusters. Cluster 4 did not significantly correlated to any primary cell type, likely due to the small number of observed cells. Notably, few hematopoietic cells (PTPRC; CD45) positive cells were observed (FIG. 10A), including CD68⁺ macrophages which share many markers with LSECs. Taken together these data suggest that hESC-derived angioblasts and LSEC-LCs are capable of engrafting neonatal NSG livers, responding to endogenous drivers of proliferation and maturation to form human LSECs with gene signatures similar to adult human LSECs.

A key functional characteristic of mature LSECs is the presence of transcellular fenestrations clustered in dynamically regulated regionalized areas of the endothelial cell surface known as sieve plates (Braet and Wisse, 2002; DeLeve, 2013b). These fenestrated sieve plates distinguish sinusoidal endothelial cells from hepatic portal vein and arterial endothelium (FIG. 11A), but more importantly also distinguish continuous, general fenestrated, and sinusoidal capillary subtypes. To test for the presence of regulated fenestrae we isolated the four subfractions examined in FIG. 7, cultured them overnight, treated them with or without the actin depolymerizing agent latrunculin A (Braet et al., 1998) for 30 minutes then processed them and control cell populations for scanning electron microscopy. In the absence of latrunculin stimulation, only tdRFP⁺ CD31⁺ CD32⁺ LYVE1⁺ cells naturally had fenestrations arranged in sieve plates which covered approximately 8% of the cell surface and were generally 90-110 nm in size similar to those observed in primary human LSECs in situ (FIG. 11B, D, F). Upon stimulation with latrunculin, both tdRFP⁺ CD31⁺ CD32⁺ LYVE1⁺ and tdRFP⁺ CD31⁺ CD32⁺ to LYVE1⁻ cells were found to have fenestrations arranged in sieve plates (FIG. 11B). In tdRFP⁺ CD31⁺ CD32⁺ LYVE1⁺ cells, the stimulated fenestrations covered approximately 40% of the cell surface with the increase attributed to a smaller ˜60 nm fenestration type that was more rarely observed without the acute stimulus (FIG. 11B, E, F). Importantly, HUVECs and tdRFP⁺CD31⁺CD32⁺LYVE1⁺ extended culture venous LSEC-LCs, representing industry standard endothelial cells and the in vitro derived venous SEC-LCs, respectively, were not naturally fenestrated nor were they able to be induced to form fenestrations with latrunculin treatment. This suggests that the in vivo maturation of hPSC-derived angioblasts to fenestrated LSECs is incompletely recapitulated in the current in vitro culture system.

To be useful as a cellular therapeutic option, engraftment and LSEC maturation of angioblasts should not be restricted to models of neonatal transplantation or requirement of whole body irradiation. To address these constraints, we adopted a surgical transplantation approach in adult NSG mice in which the irradiation model was changed for a monocrotaline preconditioning model. Here, adult (16+ week old) NSG mice were treated once with monocrotaline (150 mg/kg, IP) to induce endothelial liver injury. Twenty-four hours after injury induction, day 8 arterial or venous angioblasts as used in neonatal studies were surgically delivered via a laparotomy mediated intrasplenic injection style transplantation process. In this way, angioblasts were infused into the damaged liver via the native hepatic vasculature without surgical disturbance of the liver (FIG. 12A). To determine if adult mice were effectively engrafted before sacrifice, circulating human specific FVIII levels were assessed in the blood of multiple mouse samples (FIG. 12B). Non transplanted NSG mice and neonatal intrahepatic HUVEC transplanted mice did not have detectable human FVIII. Neonatal mice transplanted with hPSC-derived angioblast and endothelial populations did show detectable FVIII levels, but only in mice which were verified to have elevated engraftment levels (>1% RFP of NPC fraction). Similarly to the best engrafted neonatal mice, adult NSG mice following intrasplenic angioblast transplantation showed elevated FVIII levels, suggestive of efficient engraftment in adult mice despite the short time period post transplantation (30-41 days post translation), which was approximately half that of the neonatal studies.

To determine if the adult mice were indeed engrafted with maturing functional hPSC-derived LSECs, histological analysis was performed. Day 8 vein angioblast engrafted adult mice showed large areas of sinusoidal space engrafted with KU80 positive human cells which were also expressing human specific LSEC markers (CD31, CD32B, LYVE1 and STAB2) (FIG. 12C-12F). Quantitative analysis of NPC composition revealed that, while both artery and vein angioblasts are capable of engrafting adult NSG livers (100% of mice were >1% %RFP+; FIG. 12G), venous angioblasts have improved engraftment efficiency (up to 66% RFP+ of NPC), which approaches complete humanization of the liver vasculature compartment in the NPC samples (FIG. 12H). The composition of both the artery and venous angioblast-derived populations from adult transplantation studies was strongly biased to endothelial cell fractions with a further strong bias to CD31+CD32+LYVE1+ central vein-associated hPSC-derived mature LSECs similar to those seen in the neonatal studies. This adult transplantation work suggests that the neonatal liver, while permissive to engraftment, may be less optimal than the MCT-injured adult liver, and furthermore suggests a broad functional utility to the venous angioblast population to engraft and mature under diverse liver injury models.

Additional method details are provided in Examples 3 and 4.

### Example 2

The liver contains many functional cell types, all of which are perfused by an anatomically unique vasculature where both hepatic arterial and portal venous blood mix in a sinusoidal endothelium that then drains to a central vein. In addition to scavenging functions, the liver sinusoidal endothelial cell (LSEC) plays a key role in liver homeostasis by tuning regeneration and fibrosis responses by dynamic and responsive cytokine production. To facilitate future tissue engineering and to illuminate human hepatic vasculature development, we have generated angioblasts from human pluripotent stem cells (hPSCs) and examined their in vitro and in vivo developmental ability to form functional LSECs. First, hPSCs were differentiated through a common vasculogenic mesoderm then specified to angioblasts by modulation of VEGF, bFGF, and NOTCH signaling. In vitro, purified CD34⁺ angioblasts adopted a proliferative endothelial cell (EC) fate upon adherent culture and responded to TGFbeta signaling inhibition, cAMP signaling agonist, and oxygen tension by upregulation of LSEC markers. To deliver physiological cues associated with functional LSEC development, a neonatal intrahepatic transplantation model was applied. Recovery of transplanted hPSC-derived cells revealed expression of mature LSEC markers at levels seen in isolated primary human LSECs including F8 which when mutated is clinically associated with Hemophilia A. Furthermore, scRNA-seq of recovered cells showed a close transcriptional profile to primary LSECs including LSEC zonation. Functional analysis of recovered hPSC-derived LSECs revealed a high degree of correctly sized fenestrations that were dynamically responsive to actin depolymerization by latrunculin A treatment. Taken together, this work describes the in vitro differentiation of hPSC-derived venous vasculature progenitors capable of in vivo maturation to functional LSECs with future uses in toxicology with human hepatic vascularized mice or as a cellular therapy for Hemophilia A.

### Example 3

Throughout the differentiation timeline the same media can be utilized for simplicity and practicality. In Examples 1 and 2, the base media consisted of 25% v/v StemPro34, 75% v/v IMDM, supplemented with 1:10,000 ITS-X, L-glutamine (2 mM), ascorbic acid (1 mM), monothioglycerol (MTG, 4×10⁻⁴ M; Sigma), transferrin (150 μg/mL). This media was supplemented as appropriate for the stages. Day −1 to day 0, 1 ng/ml BMP4. Day 0 to day 1, 10 ng/ml BMP4. Day 1 to day 2, 10 ng/ml BMP4, 5 ng/ml bFGF. Day 2 to day 4, 10 ng/ml BMP4, 5 ng/ml bFGF, 3 microM CHIR99021. Day 4 to day 8, 10 or 100 ng/ml VEGFA, 30 ng/ml bFGF, 0 or 10 microM GSI. Day 8, 10 or 100 ng/ml VEGFA, 30 ng/ml bFGF, 10 microM Y-27632. Day 9 to day 12, 10 or 100 ng/ml VEGFA, 30 ng/ml bFGF. Day 12 to day 22, 30 ng/ml bFGF, 6 microM SB, 1 mM 8-Br-cAMP. All growth factors are from R&D systems, small molecules are from Tocris, cAMP is from Biolog.

Other base medias or combinations can be used. Commercially available StemPro34 (ThermoFisher Scientific, 10639011) can be used as supplied or partially diluted with IMDM (ThermoFisher Scientific, 12200036) further supplemented with ITS-X (ThermoFisher Scientific, 51500056) additional glutamine, ascorbic acid, monothioglycerol and transferrin. GMEM, DMEM, and RPMI can also be used with ITS-X or other supplements.

### Example 4

Preparing Embryoid bodies from PSC to Day 8 differentiation

The hESC lines such as HES2-tdRFP and H1-GFP, or human iPS cell line MSC-iPS1 can be used. Said cells can be maintained on irradiated mouse embryonic fibroblasts in hESC media as described previously (Lancrin C, et al. The haemangioblast generates haematopoietic cells through a haemogenic endothelium stage. Nature. 2009;457:892-895). For embryoid body differentiation at day −1, 85-95% confluent hPSC were dissociated to single cells (TrypLE, ThermoFisher) and re-aggregated to form EBs in base media (25% StemPro34, 75% IMDM, and supplements) with 1 ng/ml BMP4, 10 microM Y-27632 for 18 hours on an orbital shaker (5%CO₂, 5% O₂, 90% N₂). As seen in FIG. 1A, at day 0, pluripotent EBs were induced toward mesoderm by transfer to base media with 10 ng/ml BMP4. At day 1, base media was added to EBs and day 0 media to replenish 10 ng/ml BMP4 and add 5 ng/ml bFGF. On day 2, EBs passing through primitive streak were transferred to fresh base media containing 10 ng/ml BMP4, 5 ng/ml bFGF, 3 microM CHIR99021. Day 4 mesoderm EBs were transferred to base media containing 30 ng/ml bFGF and 10 ng/ml (venous) or 100 ng/ml (arterial) VEGFA with 10 microM (venous) GSI. On day 6, EBs containing angioblasts were transferred to fresh day 4 media to continue specification to day 8. On day 8 well specified angioblast containing EBs were dissociated by sequential addition of Trypsin EDTA (5 mins, 37° C.), and Collagenase Type 1 (Sigma, 60 minutes, 37° C.) followed by light trituration. Single-cell suspensions were subsequently enriched for CD34+ angioblasts by MACS (Miltenyi 130-146-702) following manufacture recommended protocols modified to stain 5 million cells with 10 microliters of CD34-mag antibody in 100 micro liters of volume.

### REFERENCES

Araiet al., 2011, Peptides, 32:1855-65

Braet et al., 1998, PNAS USA, 95:13635-40

Braet and Wisse, 2002, Comp. Hepatology, 1:1

Deleve, 2013a, In “Drug-Induced Liver Disease,” Kaplowitz and Deleve, eds., pp. 135-143

DeLeve, 2013b, J. Clin. Invest., 123:1861-6

Ding et al., 2010, Nature, 468:310-5

Ditadi et al., 2015, Nat. Cell Biol., 17:580-91

Elvevold et al., 2008, Am. J. Physiol. Gastrointest. Liver Physiol., 294:G391-400

Fomin et al., 2013, PLoS ONE, 8:e77255

Hasan et al., 2017, Nat. Cell Biol., 19:928-40

Hen et al., 2015, Development, 142:4266-78

Irion et al., 2007, Nat. Biotechnol., 25:1477-82

Koui et al., 2017, Stem Cell Reports, 9:490-498

Lautt and Greenway, 1987, Hepatology, 7:952-63

Martinez et al., 2008, Comp.Hepatology, 7:4

McCuskey, 2000, Liver, 20:3-7

McParland et al., 2018, Nat. Commun., 9:4383

Shahani et al., 2014, J. Thrombosis Haemostasis, 12:36-42

Strauss et al., 2017, Sci. Rep., 7:44356

Sturgeon et al., 2014, Nat. Biotechnol., 32:554-61

Wisse et al., 1985, Hepatology, 5:683-92

Zhang et al., 2016, Nat. Genet., 48:537-43

Zhang et al., 2017, PNAS USA, 114:E6072-8

1. A method of producing sinusoidal endothelial cell-like cells (SEC-LCs) comprising:
providing stem cells or angioblasts; and
culturing the stem cells or angioblasts under conditions in which SEC-LCs are produced, wherein the conditions comprise:
a) culturing the stem cells or angioblasts in the presence of bFGF; or
b) culturing the stem cells or angioblasts in the presence of vascular endothelial growth factor (VEGF)-A to produce endothelial cells followed by culturing the endothelial cells in the presence of a TGF-beta signaling inhibitor, cyclic AMP (cAMP) signaling agonist, VEGF-C; or
c) culturing the stem cells or angioblasts under hypoxic conditions, thereby producing SEC-LCs.

2. The method of claim 1, wherein the SEC-LCs are liver SEC-LCs.

3. The method of claim 1, wherein the stem cells are pluripotent stem cells, induced pluripotent stem cells, or embryoid bodies.

4. The method of claim 1, wherein the stem cells are cultured in the presence of BMP4, bFGF, and/or CHIR.

5. The method of claim 1, wherein the stem cells are cultured in the presence of Notch inhibitor or a MEK inhibitor, bFGF and/or a venous angioblast specifying concentration of VEGF (a venous angioblast inducing media).

6. The method of claim 1, further comprising culturing the stem cells or angioblasts in the presence of a Notch inhibitor.

7. The method of claim 1, wherein the angioblasts are venous angioblasts or arterial angioblasts.

8. The method of claim 1, wherein the cAMP signaling agonist is cAMP, 8-Br-cAMP, forskolin and/or IBMX.

9. The method of claim 1, wherein the TGFbeta signaling inhibitor is SB431542.

10. The method of claim 1, wherein the hypoxic conditions comprise culturing in the presence of 5% CO₂/5% O₂ or culturing in the presence of a hypoxia inducible factor (HIF) prolyl-hydroxylase (PHD) inhibitor (HIF-PHDI).

11. The method of claim 10, wherein the HIF-PHDI is a tricyclic triazole compound.

12. The method of claim 10, wherein the HIF-PHDI is selected from Daprodustat, Molidustat, Roxadustat, Vadadustat and Desidustat.

13. The method of claim 1, wherein the SEC-LCs are cultured in the presence of TGFbeta signaling inhibitor, a cAMP signaling agonist, and/or a deficiency in VEGF-C.

14. The method of claim 1, wherein the SEC-LCs express Factor VIII.

15. The method of claim 1, further comprising monitoring the SEC-LCs for the presence of Factor VIII.

16. The method of claim 1, further comprising isolating the SEC-LCs.

17. A population of SEC-LC cells produced by the method of claim 1.

18-21. (canceled)

22. A method of treating an individual suffering from a liver disease, comprising:
introducing the SEC-LCs of claim 17 into the individual.

23. The method of claim 22, wherein the liver disease is nonalcoholic steatohepatitis (fatty liver disease or NASH), progressive cirrhosis diseases or disorders, Hemophilia A, or hepatocellular carcinoma (HCC).

24. The method of claim 22, wherein the administering is directly to the liver.

25. (canceled)
